(19) United States
(12) Patent Application Publication
(10) Pub. No.: US 2019/0192477 A1
(43) Pub. Date: Jun. 27, 2019

(54) NANOPARTICLE COMPOSITIONS OF ALBUMIN AND PACLITAXEL

(71) Applicant: Abraxis BioScience, LLC, Summit, NJ (US)

(72) Inventor: Neil P. Desai, Pacific Palisades, CA (US)

(21) Appl. No.: 16/170,522

(22) Filed: Oct. 25, 2018

Related U.S. Application Data
(63) Continuation of application No. 13/794,705, filed on Mar. 11, 2013, now abandoned.
(60) Provisional application No. 61/747,123, filed on Dec. 28, 2012.

(51) Int. Cl. A61K 31/337; A61K 9/51

(52) U.S. Cl. CPC A61K 31/337; A61K 9/5169

(57) ABSTRACT

The present invention provides compositions (such as pharmaceutical compositions) comprising nanoparticles comprising albumin and paclitaxel. The compositions have a specific albumin polymer/monomer profile and are particularly suitable for use in treating diseases such as cancer.

### RELATED APPLICATIONS

This application is a continuation of U.S. patent application Ser. No. 13/794,705, filed Mar. 11, 2013, which claims priority from U.S. Provisional Application No. 61/747,123, filed Dec. 28, 2012, the disclosures of each of which are incorporated herein by reference in their entirety.

### TECHNICAL FIELD

The present invention relates to compositions comprising nanoparticles comprising albumin and paclitaxel.

### BACKGROUND

Albumin-based nanoparticle compositions have been developed as a drug delivery system for delivering substantially water insoluble drugs such as a taxane. See, for example, U.S. Pat. Nos. 5,916,596; 6,506,405; 6,749,868, and 6,537,579, 7,820,788, and 7,923,536. ABRAXANE®, an albumin stabilized nanoparticle formulation of paclitaxel, was approved in the United States in 2005 and subsequently in various other countries for treating metastatic breast cancer. It was recently approved for treating non-small cell lung cancer in the United States, and has also shown therapeutic efficacy in various clinical trials for treating difficult-to-treat cancers such as pancreatic cancer and melanoma. Albumin derived from human blood has been used for the manufacture of ABRAXANE® as well as various other albumin-based nanoparticle compositions.

It is generally believed that albumin-based nanoparticles, such as those in ABRAXANE®, when introduced into the blood stream, would dissolve into albumin-drug complexes. Such albumin-drug complexes utilize the natural properties of albumin to transport and deliver substantially water insoluble drugs to the site of disease, such as tumor sites. In addition, the albumin-based nanoparticle technology offers the ability to improve a drug's solubility by avoiding the need for toxic solvents in the administration process, thus potentially improving safety through the elimination of solvent-related side effects.

The disclosures of all publications, patents, patent applications and published patent applications referred to herein are hereby incorporated herein by reference in their entirety.

### BRIEF SUMMARY DESCRIBED HEREIN

The present application in some embodiment provides a composition (such as pharmaceutical composition) comprising nanoparticles comprising albumin (such as human albumin) and paclitaxel, wherein no greater than about 2.4% (such as no greater than about 1.5%, or about 0%) of the total albumin in the composition is in the form of polymers. In some embodiments, at least about 80% (such as at least about 92%) of the total albumin in the composition is in the form of monomers. In some embodiments according to any one of the compositions (such as pharmaceutical compositions) described above, no greater than about 10% of total albumin in the composition is in the form of dimers. In some embodiments, no greater than about 3% of total albumin in the composition is in the form of oligomers. The composition (such as pharmaceutical composition) described above may or may not comprise sucrose and/or edetate.

In some embodiments according to any one of the compositions (such as pharmaceutical compositions) described above, at least about 60% of the monomeric albumins in the composition have a free thiol group. In some embodiments, at least about 60% of the monomeric albumin in the composition has a blocked thiol group. In some embodiments, the composition is substantially free of albumin lacking C-terminal Leu and albumin lacking N-terminal Asp-Ala, and/or has an albumin glycosylation profile that is different from that of native albumin obtained from a human (for example in some embodiments the composition contains no glycosylated albumin). In some embodiments, the composition is substantially free of any one or more of the following: fatty acids, caprylate, tryptophan, blood component, virus, and/or prion.

In some embodiments according to any one of the compositions (such as pharmaceutical compositions) described above, no greater than about 0.5% of 7-epipaclitaxel is generated upon storage of the composition (such as pharmaceutical composition) at 55° C. for about two weeks and/or no greater than about 0.7% of 7-epipaclitaxel is generated upon storage of the composition (such as pharmaceutical composition) at 55° C. for about 1 month. In some embodiments, no greater than about 0.45% total impurities were generated upon storage of the composition (such as pharmaceutical composition) at 55° C. for about two weeks and/or no greater than about 0.65% total impurities were generated upon storage of the composition (such as pharmaceutical composition) at 55° C. for about 1 month.

In some embodiments according to any one of the compositions (such as pharmaceutical compositions) described above, no greater than about 1% additional albumin polymers are generated upon storage of the composition (such as pharmaceutical composition) at 55° C. for about two weeks, no greater than about 1% additional albumin polymers are generated upon storage of the composition (such as pharmaceutical composition) at 55° C. for about 1 month, no greater than about 10% albumin monomers are lost upon storage of the composition (such as pharmaceutical composition) at 55° C. for about two weeks, and/or no greater than about 20% albumin monomers are lost upon storage of the composition (such as pharmaceutical composition) at 55° C. for about 1 month.

In some embodiments according to any one of the compositions (such as pharmaceutical compositions) described above, at least about 80% of the total albumin in the composition is not associated with the nanoparticles. In some embodiments, the nanoparticles comprise paclitaxel coated with albumin. In some embodiments, the nanoparticles are substantially free of polymeric core matrix. In some embodiments, the nanoparticles in the composition have an average diameter of no greater than about 200 nm. In some embodiments, the weight ratio of the albumin and the paclitaxel in the composition is about 9:1 to about 1:1 (including for example about 8:1 to about 1:1). In some embodiments, the composition (such as pharmaceutical composition) has two or more (such as all) of these characteristics.

In some embodiments, there is provided a commercial batch of any one of the compositions (such as pharmaceutical compositions) described above.

In some embodiments, there is provided a method of treating a disease (such as cancer) in an individual (such as a human individual) comprising administering to the individual an effective amount of any one of the pharmaceutical compositions described above.

Also provided are kits, medicines, and articles of manufacture comprising any one of the compositions (such as pharmaceutical compositions) described above.

### DETAILED DESCRIPTION

The present application provides albumin/paclitaxel nanoparticle compositions (such as pharmaceutical compositions) having a specific albumin profile. Specifically, the albumin/paclitaxel nanoparticle compositions described herein contain no greater than about 2.4% albumin polymers, contain least about 92% of albumin monomers, and/or have a monomer/polymer weight ratio of at least about 33:1. For example, in some embodiments, no greater than about 2.4% of the total albumin in the composition is in the form of polymers. In some embodiments, at least about 92% of the total albumin in the composition is in the form of monomers. In some embodiments, no greater than about 2.4% of the total albumin in the composition is in the form of polymers and at least about 80% of the total albumin in the composition is in the form of monomers. In some embodiments, the weight ratio of monomer to polymer in the composition is at least about 33:1. In some embodiments, no greater than about 2.4% of the total albumin in the composition is in the form of polymers, and the weight ratio of monomer to polymer in the composition is at least about 33:1. In some embodiments, no greater than about 2.4% of the total albumin in the composition is in the form of polymers, at least about 80% of the total albumin in the composition is in the form of monomers, and the weight ratio of monomer to polymer in the composition is at least about 33:1. In some embodiments, the composition comprises at least about 80% albumin monomers, no greater than about 2.4% albumin polymers, no greater than about 15% (such as about 4% to about 15%, for example about 4% to about 10%) albumin dimers, and no greater than about 10% (such as no greater than about 5%, for example no greater than about 1%) albumin oligomers.

The compositions (such as pharmaceutical compositions) disclosed herein are useful for treating various diseases, such as cancer. The present application thus provides compositions (such as pharmaceutical compositions, including for example commercial batches) having a specific albumin monomer/polymer profile, as well as methods of using such composition for the treatment of diseases, including cancer. Also provided are kits, medicines, and dosage forms comprising the compositions (such as pharmaceutical compositions) described herein and for use in methods described herein.

### Definitions

The term “individual” refers to a mammal and includes, but is not limited to, human, bovine, horse, feline, canine, rodent, or primate.

It is understood that aspects and embodiments described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise.

“Monomers” used herein refers to a single albumin molecule without intermolecular disulfide bonds.

“RRT” used herein refers to the retention time relative to the albumin monomers retention on a size-exclusion HPLC chromatography.

“Dimers” used herein refers to albumin species having an RRT of about 0.86 to about 0.97.

“Oligomers” used herein refers to albumin species having an RRT of about 0.70 to about 0.85.

“Polymers” used herein refers to albumin species having an RRT of about 0.57 to about 0.69.

### Albumin/Paclitaxel Nanoparticle Compositions

The present application in some embodiments provides a composition (such as pharmaceutical composition) comprising nanoparticles comprising albumin and paclitaxel, wherein no greater than about 2.4% of the total albumin in the composition is in the form of polymers. In some embodiments there is provided a composition (such as pharmaceutical composition) comprising nanoparticles comprising paclitaxel coated with albumin, wherein no greater than about 2.4% of the total albumin in the composition is in the form of polymers. In some embodiments, there is provided a composition (such as pharmaceutical composition) comprising nanoparticles comprising paclitaxel coated with albumin, wherein the average diameter of the nanoparticles in the composition is no greater than about 200 nanometers, wherein no greater than about 2.4% of the total albumin in the composition is in the form of polymers. In some embodiments, there is provided a composition (such as pharmaceutical composition) comprising nanoparticles comprising paclitaxel coated with human albumin, wherein the average diameter of the nanoparticles in the composition is no greater than about 150 nanometers (for example about 130 nm), wherein no greater than about 2.4% of the total albumin in the composition is in the form of polymers. In some embodiments, no greater than about 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the total albumin in the composition is in the form of polymers. In some embodiments, about 0% of the total albumin in the composition is in the form of polymers. In some embodiments, at least about 60% of the monomeric albumins in the composition have a free thiol group, i.e., are not blocked by a group such as a cysteine. In some embodiments, at least about 60% of the monomeric albumins in the composition have a blocked thiol group, e.g., blocked by a cysteine. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a composition (such as pharmaceutical composition) comprising nanoparticles comprising albumin and paclitaxel, wherein at least about 92% of the total albumin in the composition is in the form of monomers. In some embodiments there is provided a composition (such as pharmaceutical composition) comprising nanoparticles comprising paclitaxel coated with albumin, wherein at least about 92% of the total albumin in the composition is in the form of monomers. In some embodiments, there is provided a composition (such as pharmaceutical composition) comprising nanoparticles comprising paclitaxel coated with albumin, wherein the average diameter of the nanoparticles in the composition is no greater than about 200 nanometers, and wherein at least about 92% of the total albumin in the composition is in the form of monomers. In some embodiments, there is provided a composition (such as pharmaceutical composition) comprising nanoparticles comprising paclitaxel coated with human albumin, wherein the average diameter of the nanoparticles in the composition is no greater than about 150 nanometers (for example about 130 nm), and wherein at least about 92% of the total albumin in the composition is in the form of monomers. In some embodiments, at least about 93%, 94%, or 95% of the total albumin in the composition is in the form of monomers. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel is about 9:1.

In some embodiments, there is provided a composition (such as pharmaceutical composition) comprising nanoparticles comprising albumin and paclitaxel, wherein the weight ratio of albumin monomers to albumin polymers in the composition is at least about 33:1. In some embodiments there is provided a composition (such as pharmaceutical composition) comprising nanoparticles comprising paclitaxel coated with albumin, wherein the weight ratio of albumin monomers to albumin polymers in the composition is at least about 33:1. In some embodiments, there is provided a composition (such as pharmaceutical composition) comprising nanoparticles comprising paclitaxel coated with albumin, wherein the average diameter of the nanoparticles in the composition is no greater than about 200 nanometers, wherein the weight ratio of albumin monomers to albumin polymers in the composition is at least about 33:1. In some embodiments, there is provided a composition (such as pharmaceutical composition) comprising nanoparticles comprising paclitaxel coated with human albumin, wherein the average diameter of the nanoparticles in the composition is no greater than about 150 nanometers (for example about 130 nm), wherein the weight ratio of albumin monomers to albumin polymers in the composition is at least about 33:1. In some embodiments, the weight ratio of albumin monomers to albumin polymers in the composition is at least about any of 34:1, 35:1, 36:1, 37:1, 38:1, 39:1, 40:1, 41:1, 42:1, 43:1, 44:1, 45:1, 46:1, 47:1, or 48:1. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a composition (such as pharmaceutical composition) comprising nanoparticles comprising albumin and paclitaxel, wherein at least about 80% of the total albumin in the composition is in the form of monomers, and wherein no greater than about 2.4% of the total albumin in the composition is in the form of polymers. In some embodiments there is provided a composition (such as pharmaceutical composition) comprising nanoparticles comprising paclitaxel coated with albumin, wherein at least about 80% of the total albumin in the composition is in the form of monomers, and wherein no greater than about 2.4% of the total albumin in the composition is in the form of polymers. In some embodiments, there is provided a composition (such as pharmaceutical composition) comprising nanoparticles comprising paclitaxel coated with albumin, wherein the average diameter of the nanoparticles in the composition is no greater than about 200 nanometers, wherein at least about 80% of the total albumin in the composition is in the form of monomers, and wherein no greater than about 2.4% of the total albumin in the composition is in the form of polymers. In some embodiments, there is provided a composition (such as pharmaceutical composition) comprising nanoparticles comprising paclitaxel coated with human albumin, wherein the average diameter of the nanoparticles in the composition is no greater than about 150 nanometers (for example about 130 nm), wherein at least about 80% of the total albumin in the composition is in the form of monomers, and wherein no greater than about 2.4% of the total albumin in the composition is in the form of polymers. In some embodiments, at least about 81%, 82%, 83%, 84%, 85%, 86%, 87%, 88%, 89%, 90%, 91%, 92%, 93%, 94%, or 95% of the total albumin in the composition is in the form of monomers. In some embodiments, no greater than about 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the total albumin in the composition is in the form of polymers. In some embodiments, about 0% of the total albumin in the composition is in the form of polymers. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a composition (such as pharmaceutical composition) comprising nanoparticles comprising albumin and paclitaxel, wherein at least about 80% of the total albumin in the composition is in the form of monomers, and wherein the weight ratio of albumin monomers to albumin polymers in the composition is at least about 33:1. In some embodiments there is provided a composition (such as pharmaceutical composition) comprising nanoparticles comprising paclitaxel coated with albumin, wherein at least about 80% of the total albumin in the composition is in the form of monomers, and wherein the weight ratio of albumin monomers to albumin polymers in the composition is at least about 33:1. In some embodiments, there is provided a composition (such as pharmaceutical composition) comprising nanoparticles comprising paclitaxel coated with albumin, wherein the average diameter of the nanoparticles in the composition is no greater than about 200 nanometers, wherein at least about 80% of the total albumin in the composition is in the form of monomers, and wherein the weight ratio of albumin monomers to albumin polymers in the composition is at least about 33:1. In some embodiments, there is provided a composition (such as pharmaceutical composition) comprising nanoparticles comprising paclitaxel coated with human albumin, wherein the average diameter of the nanoparticles in the composition is no greater than about 150 nanometers (for example about 130 nm), wherein at least about 80% of the total albumin in the composition is in the form of monomers, and wherein the weight ratio of albumin monomers to albumin polymers in the composition is at least about 33:1. In some embodiments, at least about 81%, 82%, 83%, 84%, 85%, 86%, 87%, 88%, 89%, 90%, 91%, 92%, 93%, 94%, or 95% of the total albumin in the composition is in the form of monomers. In some embodiments, the weight ratio of albumin monomers to albumin polymers in the composition is at least about any of 33:1, 34:1, 35:1, 36:1, 37:1, 38:1, 39:1, 40:1, 41:1, 42:1, 43:1, 44:1, 45:1, 46:1, 47:1, or 48:1. In some embodiments, at least about 60% of the monomeric albumins in the composition have a free thiol group, i.e., are not blocked by a cysteine. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a composition (such as pharmaceutical composition) comprising nanoparticles comprising albumin and paclitaxel, wherein at least about 80% of the total albumin in the composition is in the form of monomers, wherein no greater than about 2.4% of the total albumin in the composition is in the form of polymers, wherein no greater than about 15% (such as about 4% to about 15%, for example about 4% to about 10%) of the total albumin in the composition is in the form of dimers, and wherein no greater than about 10% (such as no greater than about 5%, for example no greater than about 1%) of the total albumin in the composition is in the form of oligomers. In some embodiments there is provided a composition (such as pharmaceutical composition) comprising nanoparticles comprising paclitaxel coated with albumin, wherein at least about 80% of the total albumin in the composition is in the form of monomers, wherein no greater than about 2.4% of the total albumin in the composition is in the form of polymers, wherein no greater than about 15% (such as about 4% to about 15%, for example about 4% to about 10%) of the total albumin in the composition is in the form of dimers, and wherein no greater than about 10% (such as no greater than about 5%, for example no greater than about 1%) of the total albumin in the composition is in the form of oligomers. In some embodiments, there is provided a composition (such as pharmaceutical composition) comprising nanoparticles comprising paclitaxel coated with albumin, wherein the average diameter of the nanoparticles in the composition is no greater than about 200 nanometers, wherein at least about 80% of the total albumin in the composition is in the form of monomers, wherein no greater than about 2.4% of the total albumin in the composition is in the form of polymers, wherein no greater than about 15% (such as about 4% to about 15%, for example about 4% to about 10%) of the total albumin in the composition is in the form of dimers, and wherein no greater than about 10% (such as no greater than about 5%, for example no greater than about 1%) of the total albumin in the composition is in the form of oligomers. In some embodiments, there is provided a composition (such as pharmaceutical composition) comprising nanoparticles comprising paclitaxel coated with human albumin, wherein the average diameter of the nanoparticles in the composition is no greater than about 150 nanometers (for example about 130 nm), wherein at least about 80% of the total albumin in the composition is in the form of monomers, wherein no greater than about 2.4% of the total albumin in the composition is in the form of polymers, wherein no greater than about 15% (such as about 4% to about 15%, for example about 4% to about 10%) of the total albumin in the composition is in the form of dimers, and wherein no greater than about 10% (such as no greater than about 5%, for example no greater than about 1%) of the total albumin in the composition is in the form of oligomers. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a composition (such as pharmaceutical composition) comprising nanoparticles comprising albumin and paclitaxel, wherein about 80% to about 95% of the total albumin in the composition is in the form of monomers, wherein about 0% to about 1.5% (such as about 0% to about 0.5%, for example 0%) of the total albumin in the composition is in the form of polymers, wherein about 4% to about 15% (such as about 4% to about 10%, for example about 5% to about 7%) of the total albumin in the composition is in the form of dimers, and wherein no greater than about 0% to about 10% (such as about 0% to about 5%, for example about 0% to about 1%, including about 0.4% to about 0.8%, about 0.5% to about 0.7%) of the total albumin in the composition is in the form of oligomers. In some embodiments there is provided a composition (such as pharmaceutical composition) comprising nanoparticles comprising paclitaxel coated with albumin, wherein about 80% to about 95% of the total albumin in the composition is in the form of monomers, wherein about 0% to about 1.5% (such as about 0% to about 0.5%, for example 0%) of the total albumin in the composition is in the form of polymers, wherein about 4% to about 15% (such as about 4% to about 10%, for example about 5% to about 7%) of the total albumin in the composition is in the form of dimers, and wherein no greater than about 0% to about 10% (such as about 0% to about 5%, for example about 0% to about 1%, including about 0.4% to about 0.8%, about 0.5% to about 0.7%) of the total albumin in the composition is in the form of oligomers. In some embodiments, there is provided a composition (such as pharmaceutical composition) comprising nanoparticles comprising paclitaxel coated with albumin, wherein the average diameter of the nanoparticles in the composition is no greater than about 200 nanometers, wherein about 80% to about 95% of the total albumin in the composition is in the form of monomers, wherein about 0% to about 1.5% (such as about 0% to about 0.5%, for example 0%) of the total albumin in the composition is in the form of polymers, wherein about 4% to about 15% (such as about 4% to about 10%, for example about 5% to about 7%) of the total albumin in the composition is in the form of dimers, and wherein no greater than about 0% to about 10% (such as about 0% to about 5%, for example about 0% to about 1%, including about 0.4% to about 0.8%, about 0.5% to about 0.7%) of the total albumin in the composition is in the form of oligomers. In some embodiments, there is provided a composition (such as pharmaceutical composition) comprising nanoparticles comprising paclitaxel coated with human albumin, wherein the average diameter of the nanoparticles in the composition is no greater than about 150 nanometers (for example about 130 nm), wherein about 80% to about 95% of the total albumin in the composition is in the form of monomers, wherein about 0% to about 1.5% (such as about 0% to about 0.5%, for example 0%) of the total albumin in the composition is in the form of polymers, wherein about 4% to about 15% (such as about 4% to about 10%, for example about 5% to about 7%) of the total albumin in the composition is in the form of dimers, and wherein no greater than about 0% to about 10% (such as about 0% to about 5%, for example about 0% to about 1%, including about 0.4% to about 0.8%, about 0.5% to about 0.7%) of the total albumin in the composition is in the form of oligomers. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

The amount of albumin monomers, dimers, and oligomers can be determined by size-exclusion chromatography. In some embodiments, the amount of polymers is based on the amount of albumin species eluted from a size-exclusion HPLC with an RRT of about 0.57 to about 0.69 (such as an RRT of 0.57 to 0.69), for example about 0.60 to about 0.65, for example about 0.63. In some embodiments, the amount of oligomers is based on the amount of albumin species eluted from a size-exclusion HPLC with an RRT of about 0.70 to about 0.85 (such as an RRT of 0.70 to 0.85), for example about 0.74 to about 0.81, for example about 0.79. In some embodiments, the amount of dimers is based on the amount of albumin species eluted from a size-exclusion HPLC with an RRT of about 0.86 to about 0.97 (such as an RRT of 0.86 to 0.97), for example about 0.87 to about 0.91, for example about 0.88. In some embodiments, the separation range for the size-exclusion HPLC is about 10,000 to about 500,000 daltons. In some embodiments, the size-exclusion HPLC is run with a TSKgel G3000 SWXL column. In some embodiments, the size-exclusion HPLC is run with a column of TOSOH TSKgel G3000 SWXL, 7.8×300 mm, 5 μm or equivalent. In some embodiments, the size-exclusion HPLC is run with a flow rate of about 1 mL/min. In some embodiments, the size-exclusion HPLC is run at ambient temperature. In some embodiments, the size-exclusion HPLC is run with a column of TOSOH TSKgel G3000 SWXL, 7.8×300 mm, 5 μm or equivalent, at a flow rate of about 1 mL/min at room temperature. In some embodiments, the size-exclusion HPLC is run under the condition as indicated in Example 1. In some embodiments, the size-exclusion HPLC is run under the condition as indicated in Example 3.

In some embodiments, the albumin used in the manufacture of the nanoparticle composition is recombinant albumin. In some embodiments, the recombinant albumin is produced by a non-animal cell, such as yeast. The composition (such as pharmaceutical composition) obtained thereby thus can be substantially free (such as free) of a blood component or an animal component. In some embodiment, the composition (such as pharmaceutical composition) obtained using a recombinant albumin is substantially free (such as free) of virus or prion.

Recombinant albumin can be processed and manipulated in a controlled manner to: 1) alter or eliminate glycosylation profiles on albumin; 2) obtain a more homogeneous population of albumin; 3) avoid components that come naturally from albumin obtained from natural sources (e.g., from human); and 4) avoid certain salts required for the purpose of purifying native albumin from animal cells. For example, the recombinant albumin in some embodiments can be substantially free (such as free) of fatty acid, sodium caprylate, and/or tryptophanate. The recombinant albumin can be substantially free of albumin lacking C-terminal Leu and/or albumin lacking N-terminal Asp-Ala. The albumin in the nanoparticle compositions described herein can have one or more of these properties. In some embodiments, the albumin in the nanoparticle compositions has none of these properties. In some embodiments, the albumin in the nanoparticle compositions has all of these properties.

Thus, for example, the present application in some embodiments provides a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the composition is substantially free of fatty acid, wherein no greater than about 2.4% of the total albumin in the composition is in the form of polymers. In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the composition is substantially free of caprylate, wherein no greater than about 2.4% of the total albumin in the composition is in the form of polymers. In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the composition is substantially free of tryptophanate, wherein no greater than about 2.4% of the total albumin in the composition is in the form of polymers. In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the composition is substantially free of albumin lacking C-terminal Leu and/or albumin lacking N-terminal Asp-Ala, wherein no greater than about 2.4% of the total albumin in the composition is in the form of polymers. In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the albumin in the composition (such as pharmaceutical composition) has a glycosylation profile that is different from that of albumin obtained from natural sources (e.g., from human), wherein no greater than about 2.4% of the total albumin in the composition is in the form of polymers. In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the albumin in the composition (such as pharmaceutical composition) has no glycosylation, wherein no greater than about 2.4% of the total albumin in the composition is in the form of polymers. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel is about 9:1.

In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the composition is substantially free of fatty acid, caprylate, and/or tryptophanate, and wherein no greater than about 2.4% of the total albumin in the composition is in the form of polymers. In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the composition is substantially free of fatty acid, caprylate, and/or tryptophanate, wherein the composition is substantially free of albumin lacking C-terminal Leu and/or albumin lacking N-terminal Asp-Ala, and wherein no greater than about 2.4% of the total albumin in the composition is in the form of polymers. In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the composition is substantially free of fatty acid, caprylate, and/or tryptophanate, wherein the albumin in the composition (such as pharmaceutical composition) has a glycosylation profile that is different from that of albumin obtained from natural sources (e.g., from human), and wherein no greater than about 2.4% of the total albumin in the composition is in the form of polymers. In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the composition is substantially free of fatty acid, caprylate, and/or tryptophanate, wherein the albumin in the composition (such as pharmaceutical composition) has no glycosylation, wherein no greater than about 2.4% of the total albumin in the composition is in the form of polymers. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel is about 9:1.

In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the composition is substantially free of albumin lacking C-terminal Leu and/or albumin lacking N-terminal Asp-Ala, wherein the albumin in the composition (such as pharmaceutical composition) has a glycosylation profile that is different from that of albumin obtained from natural sources (e.g., from human), and wherein no greater than about 2.4% of the total albumin in the composition is in the form of polymers. In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the composition is substantially free of albumin lacking C-terminal Leu and/or albumin lacking N-terminal Asp-Ala, wherein the albumin in the composition (such as pharmaceutical composition) has no glycosylation, and wherein no greater than about 2.4% of the total albumin in the composition is in the form of polymers. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel is about 9:1.

In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the composition is substantially free of fatty acid, caprylate, and/or tryptophanate, wherein the composition is substantially free of albumin lacking C-terminal Leu and/or albumin lacking N-terminal Asp-Ala, wherein the albumin in the composition (such as pharmaceutical composition) has a glycosylation profile that is different from that of albumin obtained from natural sources (e.g., from human), and wherein no greater than about 2.4% of the total albumin in the composition is in the form of polymers. In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the composition is substantially free of fatty acid, caprylate, and/or tryptophanate, wherein the composition is substantially free of albumin lacking C-terminal Leu and/or albumin lacking N-terminal Asp-Ala, wherein the albumin in the composition (such as pharmaceutical composition) has no glycosylation, and wherein no greater than about 2.4% of the total albumin in the composition is in the form of polymers. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel is about 9:1.

In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the amount of monomer in the composition is at least about 1% (such as at least any of 1.5%, 2%, 2.5%, 3%, 4%, or 5%) more than the amount of monomer in ABRAXANE® under the same assay conditions. In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein the amount of the polymers in the composition is at least 1% (such as at least any of 1.5%, 2%, 2.5%, 3%, 4%, or 5%) less than the amount of polymers in ABRAXANE® under the same assay conditions. In some embodiments, the composition (such as pharmaceutical composition) does not comprise sucrose. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel is about 9:1.

During the storage of ABRAXANE®, impurities such as 7-epitaxel are generated over time. According to the USP monograph for paclitaxel, the acceptable upper limit of 7-epipaclitaxel present in a paclitaxel-containing composition is 0.5%. The rate of impurity generation thus affects the shelf life of the paclitaxel/albumin nanoparticle composition.

Similarly, albumin monomers in a paclitaxel/albumin nanoparticle formulation have a tendency to react or combine to form dimers, oligomers, and polymers upon storage. Increased levels of albumin dimers, oligomers, and polymers formed upon storage could cause undesirable responses in humans, such as rashes, urticaria, allergic responses, and possibly immune responses. Increased levels of albumin dimers, oligomers, and polymers in the formulation may also render the formulation susceptible to aggregation, which could affect the physical stability of the formulation.

The compositions (such as pharmaceutical compositions) provided herein in some embodiments have substantially improved impurity and albumin profiles, having a decreased rate of impurity generation and/or albumin polymerization as compared to ABRAXANE®. Such decreased rate can be assessed, for example, under accelerated conditions such as storage at 55° C. Thus, in some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein no greater than about 2.4% of the total albumin in the composition is in the form of polymers, wherein no greater than about 0.45% (such as no greater than about any of 0.4%, 0.3%, or 0.2%) of total impurities is generated upon storage of the composition (such as pharmaceutical composition) at 55° C. for about two weeks. In some embodiments, no greater than about 0.65% (such as no greater than about any of 0.6%, 0.5%, 0.4%, or 0.3%) of total impurities is generated upon storage of the composition (such as pharmaceutical composition) at 55° C. for about 1 month.

In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein no greater than about 2.4% of the total albumin in the composition is in the form of polymers, wherein no greater than about 0.5% (such as no greater than about 0.4%, 0.3%, or 0.2%) of 7-epitaxel is generated upon storage of the composition (such as pharmaceutical composition) at 55° C. for about two weeks. In some embodiments, no greater than about 0.7% (such as no greater than about 0.6%, 0.5%, or 0.4%) of 7-epitaxel is generated upon storage of the composition (such as pharmaceutical composition) at 55° C. for about 1 month. In some embodiments, the composition comprises albumin not associated with the nanoparticles.

In some embodiments, there is provided a composition (such as pharmaceutical composition) comprising nanoparticles comprising albumin and paclitaxel (such as nanoparticles comprising paclitaxel coated with albumin and/or having an average diameter of no greater than about 200 nm, for example no greater than about 150 nm), wherein no greater than about 2.4% of the total albumin in the composition is in the form of polymers, wherein no greater than about 0.3% (such as no greater than about 0.2%, 0.1%, or 0.05%) of albumin polymers is generated upon storage of the composition (such as pharmaceutical composition) at 55° C. for about two weeks. In some embodiments, no greater than about 0.4% (such as no greater than about 0.3%, 0.2%, or 0.1%) of albumin polymer is generated upon storage of the composition (such as pharmaceutical composition) at 55° C. for about 1 month. In some embodiments, the composition comprises albumin not associated with the nanoparticles.

In some embodiments, the composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 20 to about 400 nm. In some embodiments, the average or mean diameter of the nanoparticles is about 40 to about 200 nm. In some embodiments, the average or mean diameter of the nanoparticles is about 50-150 nm. In some embodiments, the nanoparticles are no less than about 50 nm. In some embodiments, the nanoparticles are sterile-filterable.

In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of the nanoparticles in the composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of the nanoparticles in the composition fall within the range of about 20 to about 400 nm, including for example about 20 to about 200 nm, about 40 to about 200 nm, about 30 to about 180 nm, and any one of about 40 to about 150, about 50 to about 120, and about 60 to about 100 nm.

In some embodiments, the nanoparticles comprise the paclitaxel coated with an albumin. In some embodiments, the composition comprises paclitaxel in both nanoparticle and non-nanoparticle forms, wherein at least about any one of 50%, 60%, 70%, 80%, 90%, 95%, or 99% of the paclitaxel in the composition are in nanoparticle form. In some embodiments, the paclitaxel in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of paclitaxel that is substantially free of polymeric materials (such as polymeric matrix).

In some embodiments, at least about any one of 50%, 60%, 70%, 80%, 90%, 95%, or 99% of the albumin in the composition are in non-nanoparticle portion of the composition.

In some embodiments, the weight ratio of albumin (such as human albumin) and a paclitaxel in the nanoparticle composition is about 18:1 or less, such as about 15:1 or less, for example about 10:1 or less. In some embodiments, the weight ratio of albumin (such as human albumin) and paclitaxel in the composition falls within the range of any one of about 1:1 to about 18:1, about 2:1 to about 15:1, about 3:1 to about 13:1, about 4:1 to about 12:1, about 5:1 to about 10:1. In some embodiments, the weight ratio of albumin and paclitaxel in the nanoparticle portion of the composition is about any one of 2:1, 3:1, 4:1, 5:1, 6:1, 7:1, 8:1, 9:1, 10:1, 15:1, or less. In some embodiments, the weight ratio of the albumin (such as human albumin) and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the nanoparticle composition comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like. In some embodiments, the composition is in sterile, lyophilized powder. In some embodiments, the composition is reconstituted with a buffer. For example, the composition (such as pharmaceutical composition) can be reconstituted in a sodium chloride buffer, such as a 0.9% sodium chloride buffer. In some embodiments, the reconstituted composition (such as pharmaceutical composition) has about 5 mg/ml of paclitaxel. In some embodiments, the composition is substantially free (for example free) of organic solvent.

Paclitaxel used herein can be obtained from a whole plant such as Taxus media, or it can be semi-synthesized. The composition (such as pharmaceutical composition) of the present application in some embodiments comprises whole plant produced paclitaxel. In some embodiments, the composition (such as pharmaceutical composition) comprises paclitaxel that is semisynthesized.

The albumin in the composition generally serves as a carrier for the paclitaxel, i.e., the albumin in the composition makes the paclitaxel more readily suspendable in an aqueous medium or helps maintain the suspension as compared to compositions not comprising an albumin. This can avoid the use of toxic solvents (or surfactants) for solubilizing the paclitaxel, and thereby can reduce one or more side effects of administration of the paclitaxel into an individual (such as a human). Thus, in some embodiments, the composition described herein is substantially free (such as free) of surfactants, such as Cremophor (including Cremophor EL® (BASF)). A composition is “substantially free of Cremophor” or “substantially free of surfactant” if the amount of Cremophor or surfactant in the composition is not sufficient to cause one or more side effect(s) in an individual when the nanoparticle composition is injected to the individual. In some embodiments, the nanoparticle composition contains less than about any one of 20%, 15%, 10%, 7.5%, 5%, 2.5%, or 1% organic solvent or surfactant.

The amount of albumin in the composition described herein will vary depending on other components in the composition. In some embodiments, the composition comprises an albumin in an amount that is sufficient to stabilize the paclitaxel in an aqueous suspension, for example, in the form of a stable colloidal suspension (such as a stable suspension of nanoparticles). In some embodiments, the albumin is in an amount that reduces the sedimentation rate of the paclitaxel in an aqueous medium. The amount of the albumin may depend on the size and density of nanoparticles of the paclitaxel.

A paclitaxel is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as human). Stability of the suspension is generally (but not necessarily) evaluated at a storage temperature (such as room temperature (such as 20-25° C.) or refrigerated conditions (such as 4° C.). For example, a suspension is stable at a storage temperature if it exhibits no flocculation or particle agglomeration visible to the naked eye or when viewed under the optical microscope at 1000 times, at about fifteen minutes after preparation of the suspension. Stability can also be evaluated under accelerated testing conditions, such as at a temperature that is higher than about 40° C. (for example 55° C.).

In some embodiments, the albumin is present in an amount that is sufficient to stabilize the paclitaxel in an aqueous suspension at a certain concentration. For example, the concentration of the paclitaxel in the composition is about 0.1 to about 100 mg/ml, including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml, about 5-15 mg/ml. In some embodiments, the concentration of the paclitaxel is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the composition, in liquid form, comprises from about 0.1% to about 50% (w/v) (e.g. about 0.5% (w/v), about 5% (w/v), about 10% (w/v), about 15% (w/v), about 20% (w/v), about 30% (w/v), about 40% (w/v), or about 50% (w/v)) of albumin. In some embodiments, the composition, in liquid form, comprises about 0.5% to about 5% (w/v) of albumin.

In some embodiments, the weight ratio of albumin, e.g., albumin, to the paclitaxel in the nanoparticle composition is such that a sufficient amount of paclitaxel binds to, or is transported by, the cell. While the weight ratio of albumin to paclitaxel will have to be optimized for different albumin and paclitaxel combinations, generally the weight ratio of albumin, e.g., albumin, to paclitaxel (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to paclitaxel weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human albumin) to the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the albumin allows the composition to be injected to an individual (such as human) without significant side effects. In some embodiments, the albumin is in an amount that is effective to reduce one or more side effects of administration of the paclitaxel to a human. The term “reducing one or more side effects of administration of the paclitaxel” refers to reduction, alleviation, elimination, or avoidance of one or more undesirable effects caused by the paclitaxel, as well as side effects caused by delivery vehicles (such as solvents that render the paclitaxel suitable for injection) used to deliver the paclitaxel. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with paclitaxel can be reduced.

In some embodiments, the nanoparticle compositions described herein comprises nanoparticles comprising paclitaxel and an albumin, wherein the nanoparticles have an average diameter of no greater than about 200 nm. In some embodiments, the nanoparticle compositions described herein comprises nanoparticles comprising paclitaxel and an albumin, wherein the nanoparticles have an average diameter of no greater than about 150 nm. In some embodiments, the nanoparticle compositions described herein comprises nanoparticles comprising paclitaxel and an albumin, wherein the nanoparticles have an average diameter of about 130 nm. In some embodiments, the nanoparticle compositions described herein comprises nanoparticles comprising paclitaxel and human albumin, wherein the nanoparticles have an average diameter of about 130 nm.

In some embodiments, the nanoparticle compositions described herein comprises nanoparticles comprising paclitaxel and an albumin (such as human albumin), wherein the nanoparticles have an average diameter of no greater than about 200 nm, wherein the weight ratio of the albumin and the taxane in the composition is no greater than about 9:1 (such as about 9:1). In some embodiments, the nanoparticle compositions described herein comprises nanoparticles comprising paclitaxel and an albumin (such as human albumin), wherein the nanoparticles have an average diameter of no greater than about 150 nm, wherein the weight ratio of the albumin and the paclitaxel in the composition is no greater than about 9:1 (such as about 9:1). In some embodiments, the nanoparticle compositions described herein comprises nanoparticles comprising paclitaxel and an albumin (such as human albumin), wherein the nanoparticles have an average diameter of about 150 nm, wherein the weight ratio of the albumin and the paclitaxel in the composition is no greater than about 9:1 (such as about 9:1). In some embodiments, the nanoparticle compositions described herein comprises nanoparticles comprising paclitaxel and human albumin, wherein the nanoparticles have an average diameter of about 130 nm, wherein the weight ratio of albumin and the taxane in the composition is about 9:1.

In some embodiments, the nanoparticle compositions described herein comprises nanoparticles comprising paclitaxel coated with an albumin (such as human albumin). In some embodiments, the nanoparticle compositions described herein comprises nanoparticles comprising paclitaxel coated with an albumin (such as human albumin), wherein the nanoparticles have an average diameter of no greater than about 200 nm. In some embodiments, the nanoparticle compositions described herein comprises nanoparticles comprising paclitaxel coated with an albumin (such as human albumin), wherein the nanoparticles have an average diameter of no greater than about 150 nm. In some embodiments, the nanoparticle compositions described herein comprises nanoparticles comprising paclitaxel coated with an albumin (such as human albumin), wherein the nanoparticles have an average diameter of about 130 nm. In some embodiments, the nanoparticle compositions described herein comprises nanoparticles comprising paclitaxel coated with human albumin, wherein the nanoparticles have an average diameter of about 130 nm.

In some embodiments, the nanoparticle compositions described herein comprises nanoparticles comprising paclitaxel coated with an albumin (such as human albumin), wherein the weight ratio of the albumin and the paclitaxel in the composition is no greater than about 9:1 (such as about 9:1). In some embodiments, the nanoparticle compositions described herein comprises nanoparticles comprising paclitaxel coated with an albumin (such as human albumin), wherein the nanoparticles have an average diameter of no greater than about 200 nm, wherein the weight ratio of the albumin and the paclitaxel in the composition is no greater than about 9:1 (such as about 9:1). In some embodiments, the nanoparticle compositions described herein comprises nanoparticles comprising paclitaxel coated with an albumin (such as human albumin), wherein the nanoparticles have an average diameter of no greater than about 150 nm, wherein the weight ratio of the albumin and the paclitaxel in the composition is no greater than about 9:1 (such as about 9:1). In some embodiments, the nanoparticle compositions described herein comprises nanoparticles comprising paclitaxel coated with an albumin (such as human albumin), wherein the nanoparticles have an average diameter of about 150 nm, wherein the weight ratio of the albumin and the paclitaxel in the composition is no greater than about 9:1 (such as about 9:1). In some embodiments, the nanoparticle compositions described herein comprises nanoparticles comprising paclitaxel coated with human albumin, wherein the nanoparticles have an average diameter of about 130 nm, wherein the weight ratio of albumin and the paclitaxel in the composition is about 9:1.

In some embodiments, the nanoparticle compositions described herein comprises nanoparticles comprising paclitaxel stabilized by an albumin (such as human albumin). In some embodiments, the nanoparticle compositions described herein comprises nanoparticles comprising paclitaxel stabilized by an albumin (such as human albumin), wherein the nanoparticles have an average diameter of no greater than about 200 nm. In some embodiments, the nanoparticle compositions described herein comprises nanoparticles comprising paclitaxel stabilized by an albumin (such as human albumin), wherein the nanoparticles have an average diameter of no greater than about 150 nm. In some embodiments, the nanoparticle compositions described herein comprises nanoparticles comprising paclitaxel stabilized by an albumin (such as human albumin), wherein the nanoparticles have an average diameter of about 130 nm. In some embodiments, the nanoparticle compositions described herein comprises nanoparticles comprising paclitaxel stabilized by human albumin, wherein the nanoparticles have an average diameter of about 130 nm.

In some embodiments, the nanoparticle compositions described herein comprises nanoparticles comprising paclitaxel stabilized by an albumin (such as human albumin), wherein the weight ratio of the albumin and the paclitaxel in the composition is no greater than about 9:1 (such as about 9:1). In some embodiments, the nanoparticle compositions described herein comprises nanoparticles comprising paclitaxel stabilized by an albumin (such as human albumin), wherein the nanoparticles have an average diameter of no greater than about 200 nm, wherein the weight ratio of the albumin and the paclitaxel in the composition is no greater than about 9:1 (such as about 9:1). In some embodiments, the nanoparticle compositions described herein comprises nanoparticles comprising paclitaxel stabilized by an albumin (such as human albumin), wherein the nanoparticles have an average diameter of no greater than about 150 nm, wherein the weight ratio of the albumin and the paclitaxel in the composition is no greater than about 9:1 (such as about 9:1). In some embodiments, the nanoparticle compositions described herein comprises nanoparticles comprising paclitaxel stabilized by an albumin (such as human albumin or human serum albumin), wherein the nanoparticles have an average diameter of about 150 nm, wherein the weight ratio of the albumin and the paclitaxel in the composition is no greater than about 9:1 (such as about 9:1). In some embodiments, the nanoparticle compositions described herein comprises nanoparticles comprising paclitaxel stabilized by human albumin, wherein the nanoparticles have an average diameter of about 130 nm, wherein the weight ratio of albumin and the paclitaxel in the composition is about 9:1.

In some embodiments, the drug exposure (AUCs) of the composition is dose proportional over about 80 to about 375 mg/m² (for example when administered with a 30 minute infusion). In some embodiments, the pharmacokinetics of paclitaxel for the composition is independent of the duration of administration. In some embodiments, the composition (such as pharmaceutical composition), when administered at a dose of 260 mg/m², has a mean maximum concentration of about 1800-2000 ng/ml (for example about 18741 ng/ml). In some embodiments, the mean total clearance of the composition (such as pharmaceutical composition) was about 15 L/hr/m2. In some embodiments, the mean volume of distribution of the composition is about 632 L/m2.

### Other Components in Compositions

In some embodiments, the compositions described herein also includes an antimicrobial agent (e.g., an agent in addition to the paclitaxel) in an amount sufficient to significantly inhibit (e.g., delay, reduce, slow, and/or prevent) microbial growth in the composition for use in the methods of treatment, methods of administration, and dosage regimes described herein. Exemplary microbial agents and variations for the use of microbial agents are disclosed in U.S. Pat. App. Pub. No. 2007/0117744A1 (such as those described in paragraphs [0036] to [0058] therein), the content of which is hereby incorporated by reference in its entirety. In some embodiments, the antimicrobial agent is a chelating agent, such as EDTA, edetate, citrate, pentetate, tromethamine, sorbate, ascorbate, derivatives thereof, or mixtures thereof. In some embodiments, the antimicrobial agent is a polydentate chelating agent. In some embodiments, the antimicrobial agent is a non-chelating agent, such as any of sulfites, benzoic acid, benzyl alcohol, chlorobutanol, and paraben. In some embodiments, an antimicrobial other than the taxane discussed above is not contained or used in the methods of treatment, methods of administration, and dosage regimes described herein.

In some embodiments, the compositions described herein include a sugar. Exemplary sugars and variations for the use of sugars are disclosed in U.S. Pat. App. Pub. No. 2007/0117744A1 (such as those described in paragraphs [0084] to [0090] therein), the content of which is hereby incorporated by reference in its entirety. In some embodiments, the sugar serves as a reconstitution enhancer which causes a lyophilized composition to dissolve or suspend in water and/or aqueous solution more quickly than the lyophilized composition would dissolve without the sugar. In some embodiments, the composition is a liquid (e.g., aqueous) composition obtained by reconstituting or resuspending a dry composition. In some embodiments, the concentration of sugar in the composition is greater than about 50 mg/ml. In some embodiments, the sugar is in an amount that is effective to increase the stability of the paclitaxel in the composition as compared to a composition without the sugar. In some embodiments, the sugar is in an amount that is effective to improve filterability of the composition as compared to a composition without the sugar.

The sugar-containing compositions described herein may further comprise one or more antimicrobial agents, such as the antimicrobial agents described herein or in U.S. Pat. App. Pub. No. 2007/0117744A1. In addition to one or more sugars, other reconstitution enhancers (such as those described in U.S. Pat. App. Publication No. 2005/0152979, which is hereby incorporated by reference in its entirety) can also be added to the compositions.

Thus, for example, the present application in some embodiments provides a composition (such as pharmaceutical composition) comprising nanoparticles comprising albumin and paclitaxel, wherein the composition further comprises sucrose and/or an edetate, wherein no greater than about 2.4% of the total albumin in the composition (such as pharmaceutical composition) is in the form of polymers. In some embodiments there is provided a composition (such as pharmaceutical composition) comprising nanoparticles comprising paclitaxel coated with albumin, wherein the composition further comprises sucrose and/or an edetate, and wherein no greater than about 2.4% of the total albumin in the composition such as pharmaceutical composition) is in the form of polymers. In some embodiments, there is provided a composition (such as pharmaceutical composition) comprising nanoparticles comprising paclitaxel coated with albumin, wherein the average diameter of the nanoparticles in the composition is no greater than about 200 nanometers, wherein no greater than about 2.4% of the total albumin in the composition (such as pharmaceutical composition) is in the form of polymers. In some embodiments, there is provided a composition (such as pharmaceutical composition) comprising nanoparticles comprising paclitaxel coated with albumin, wherein the average diameter of the nanoparticles in the composition is no greater than about 150 nanometers (for example about 130 nm), wherein the composition further comprises sucrose and/or an edetate, and wherein no greater than about 2.4% of the total albumin in the composition is in the form of polymers. In some embodiments, no greater than about 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the total albumin in the composition (such as pharmaceutical composition) is in the form of polymers. In some embodiments, about 0% of the total albumin in the composition (such as pharmaceutical composition) is in the form of polymers. In some embodiments, at least about 60% of the monomeric albumins in the composition (such as pharmaceutical composition) have a free thiol group. In some embodiments, at least about 60% of the monomeric albumins in the composition (such as pharmaceutical composition) have a blocked thiol group. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a composition (such as pharmaceutical composition) comprising nanoparticles comprising albumin and paclitaxel, wherein the composition further comprises sucrose and/or an edetate, wherein at least about 92% of the total albumin in the composition is in the form of monomers. In some embodiments there is provided a composition (such as pharmaceutical composition) comprising nanoparticles comprising paclitaxel coated with albumin, wherein the composition further comprises sucrose and/or an edetate, and wherein at least about 92% of the total albumin in the composition is in the form of monomers. In some embodiments, there is provided a composition (such as pharmaceutical composition) comprising nanoparticles comprising paclitaxel coated with albumin, wherein the average diameter of the nanoparticles in the composition is no greater than about 200 nanometers, wherein the composition further comprises sucrose and/or an edetate, and wherein at least about 92% of the total albumin in the composition is in the form of monomers. In some embodiments, there is provided a composition (such as pharmaceutical composition) comprising nanoparticles comprising paclitaxel coated with human albumin, wherein the average diameter of the nanoparticles in the composition is no greater than about 150 nanometers (such as about 130 nm), wherein the composition further comprises sucrose and/or an edetate, and wherein at least about 92% of the total albumin in the composition is in the form of monomers. In some embodiments, at least about 93%, 94%, or 95% of the total albumin in the composition is in the form of monomers. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a composition (such as pharmaceutical composition) comprising nanoparticles comprising albumin and paclitaxel, wherein the composition further comprises sucrose and/or an edetate, wherein the weight ratio of albumin monomers to albumin polymers in the composition is at least about 33:1. In some embodiments there is provided a composition (such as pharmaceutical composition) comprising nanoparticles comprising paclitaxel coated with albumin, wherein the composition further comprises sucrose and/or an edetate, and wherein the weight ratio of albumin monomers to albumin polymers in the composition is at least about 33:1. In some embodiments, there is provided a composition (such as pharmaceutical composition) comprising nanoparticles comprising paclitaxel coated with albumin, wherein the average diameter of the nanoparticles in the composition is no greater than about 200 nanometers, wherein the composition further comprises sucrose and/or an edetate, wherein the weight ratio of albumin monomers to albumin polymers in the composition is at least about 33:1. In some embodiments, there is provided a composition (such as pharmaceutical composition) comprising nanoparticles comprising paclitaxel coated with albumin, wherein the average diameter of the nanoparticles in the composition is no greater than about 200 nanometers, wherein the composition further comprises sucrose and/or an edetate, wherein the weight ratio of albumin monomers to albumin polymers in the composition is at least about 33:1. In some embodiments, there is provided a composition (such as pharmaceutical composition) comprising nanoparticles comprising paclitaxel coated with human albumin, wherein the average diameter of the nanoparticles in the composition is no greater than about 150 nanometers (such as about 130 nm), wherein the composition further comprises sucrose and/or an edetate, wherein the weight ratio of albumin monomers to albumin polymers in the composition is at least about 33:1. In some embodiments, the weight ratio of albumin monomers to albumin polymers in the composition is at least about any of 34:1, 35:1, 36:1, 37:1, 38:1, 39:1, 40:1, 41:1, 42:1, 43:1, 44:1, 45:1, 46:1, 47:1, or 48:1. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a composition (such as pharmaceutical composition) comprising nanoparticles comprising albumin and paclitaxel, wherein the composition further comprises sucrose and/or an edetate, wherein at least about 80% of the total albumin in the composition is in the form of monomers, and wherein no greater than about 2.4% of the total albumin in the composition is in the form of polymers. In some embodiments there is provided a composition (such as pharmaceutical composition) comprising nanoparticles comprising paclitaxel coated with albumin, wherein the composition further comprises sucrose and/or an edetate, wherein at least about 80% of the total albumin in the composition is in the form of monomers, and wherein no greater than about 2.4% of the total albumin in the composition is in the form of polymers. In some embodiments, there is provided a composition (such as pharmaceutical composition) comprising nanoparticles comprising paclitaxel coated with albumin, wherein the average diameter of the nanoparticles in the composition is no greater than about 200 nanometers, wherein the composition further comprises sucrose and/or an edetate, wherein at least about 80% of the total albumin in the composition is in the form of monomers, and wherein no greater than about 2.4% of the total albumin in the composition is in the form of polymers. In some embodiments, there is provided a composition (such as pharmaceutical composition) comprising nanoparticles comprising paclitaxel coated with human albumin, wherein the average diameter of the nanoparticles in the composition is no greater than about 150 nanometers (such as about 130 nm), wherein the composition further comprises sucrose and/or an edetate, wherein at least about 80% of the total albumin in the composition is in the form of monomers, and wherein no greater than about 2.4% of the total albumin in the composition is in the form of polymers. In some embodiments, at least about 81%, 82%, 83%, 84%, 85%, 86%, 87%, 88%, 89%, 90%, 91%, 92%, 93%, 94%, or 95% of the total albumin in the composition is in the form of monomers. In some embodiments, no greater than about 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the total albumin in the composition is in the form of polymers. In some embodiments, about 0% of the total albumin in the composition is in the form of polymers. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a composition (such as pharmaceutical composition) comprising nanoparticles comprising albumin and paclitaxel, wherein the composition further comprises sucrose and/or an edetate, wherein at least about 80% of the total albumin in the composition is in the form of monomers, wherein no greater than about 2.4% of the total albumin in the composition is in the form of polymers, wherein no greater than about 15% (such as about 4% to about 15%, for example about 4% to about 10%) of the total albumin in the composition is in the form of dimers, and wherein no greater than about 10% (such as no greater than about 5%, for example no greater than about 1%) of the total albumin in the composition is in the form of oligomers. In some embodiments there is provided a composition (such as pharmaceutical composition) comprising nanoparticles comprising paclitaxel coated with albumin, wherein the composition further comprises sucrose and/or an edetate, wherein at least about 80% of the total albumin in the composition is in the form of monomers, wherein no greater than about 2.4% of the total albumin in the composition is in the form of polymers, wherein no greater than about 15% (such as about 4% to about 15%, for example about 4% to about 10%) of the total albumin in the composition is in the form of dimers, and wherein no greater than about 10% (such as no greater than about 5%, for example no greater than about 1%) of the total albumin in the composition is in the form of oligomers. In some embodiments, there is provided a composition (such as pharmaceutical composition) comprising nanoparticles comprising paclitaxel coated with albumin, wherein the average diameter of the nanoparticles in the composition is no greater than about 200 nanometers, wherein the composition further comprises sucrose and/or an edetate, wherein at least about 80% of the total albumin in the composition is in the form of monomers, wherein no greater than about 2.4% of the total albumin in the composition is in the form of polymers, wherein no greater than about 15% (such as about 4% to about 15%, for example about 4% to about 10%) of the total albumin in the composition is in the form of dimers, and wherein no greater than about 10% (such as no greater than about 5%, for example no greater than about 1%) of the total albumin in the composition is in the form of oligomers. In some embodiments, there is provided a composition (such as pharmaceutical composition) comprising nanoparticles comprising paclitaxel coated with human albumin, wherein the average diameter of the nanoparticles in the composition is no greater than about 150 nanometers (such as about 130 nm), wherein the composition further comprises sucrose and/or an edetate, wherein at least about 80% of the total albumin in the composition is in the form of monomers, wherein no greater than about 2.4% of the total albumin in the composition is in the form of polymers, wherein no greater than about 15% (such as about 4% to about 15%, for example about 4% to about 10%) of the total albumin in the composition is in the form of dimers, and wherein no greater than about 10% (such as no greater than about 5%, for example no greater than about 1%) of the total albumin in the composition is in the form of oligomers. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

### Pharmaceutical Compositions and Commercial Batches

The compositions described herein may be used in pharmaceutical compositions or formulations, by combining the nanoparticle composition(s) described with a pharmaceutical acceptable carrier, excipients, stabilizing agents and/or other agents, which are known in the art, for use in the methods of treatment, methods of administration, and dosage regimes described herein.

To increase stability by increasing the negative zeta potential of nanoparticles, certain negatively charged components may be added. Such negatively charged components include, but are not limited to bile salts, bile acids, glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid, and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine, stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearoylphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

Suitable pharmaceutical carriers include sterile water; saline, dextrose; dextrose in water or saline; condensation products of castor oil and ethylene oxide combining about 30 to about 35 moles of ethylene oxide per mole of castor oil; liquid acid; lower alkanols; oils such as corn oil; peanut oil, sesame oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethylcellulose; sodium alginate; poly(vinylpyrolidone); and the like, alone, or with suitable dispensing agents such as lecithin; polyoxyethylene stearate; and the like. The carrier may also contain adjuvants such as preserving stabilizing, wetting, emulsifying agents and the like together with the penetration enhancer. The final form may be sterile and may also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

The nanoparticle compositions described herein may include other agents, excipients, or stabilizers to improve properties of the composition. Examples of suitable excipients and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate and mineral oil. The formulations can additionally include lubricating agents, wetting agents, emulsifying and suspending agents, preserving agents, sweetening agents or flavoring agents. Examples of emulsifying agents include tocopherol esters such as tocopheryl polyethylene glycol succinate and the like, Pluronic®, emulsifiers based on polyoxy ethylene compounds, Span 80 and related compounds and other emulsifiers known in the art and approved for use in animals or human dosage forms. The compositions can be formulated so as to provide rapid, sustained or delayed release of the active ingredient after administration to the patient by employing procedures well known in the art.

In some embodiments, the composition is formulated to have a pH in the range of about 4.5 to about 9.0, including for example pH ranges of any one of about 5.0 to about 8.0, about 6.5 to about 7.5, and about 6.5 to about 7.0. In some embodiments, the pH of the composition is formulated to no less than about 6, including for example no less than about any one of 6.5, 7, or 8 (e.g., about 8). The composition can also be made to be isotonic with blood by the addition of a suitable tonicity modifier, such as glycerol.

In some embodiments, the composition is suitable for administration to a human. In some embodiments, the composition is suitable for administration to a human by parenteral administration. Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizing agents, and preservatives. The formulations can be presented in unit-dose or multi-dose sealed containers, such as ampules and vials, and can be stored in a freeze-dried (lyophilized) condition requiring only the addition of the sterile liquid excipient methods of treatment, methods of administration, and dosage regimes described herein (i.e., water) for injection, immediately prior to use. Extemporaneous injection solutions and suspensions can be prepared from sterile powders, granules, and tablets of the kind previously described. Injectable formulations are preferred. In some embodiments, the composition is contained in a single-use vial, such as a single-use sealed vial. In some embodiments, each single-use vial contains about 100 mg paclitaxel. In some embodiments, the single-use vial contains about 900 mg albumin. In some embodiments, the composition is contained in a multi-use vial. In some embodiments, the composition is contained in bulk in a container.

Also provided are unit dosage forms comprising the compositions and formulations described herein. These unit dosage forms can be stored in a suitable packaging in single or multiple unit dosages and may also be further sterilized and sealed. In some embodiments, the composition (such as pharmaceutical composition) also includes one or more other compounds (or pharmaceutically acceptable salts thereof) that are useful for treating cancer. In various variations, the amount of paclitaxel in the composition is included in any one of the following ranges: about 5 to about 50 mg, about 20 to about 50 mg, about 50 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of paclitaxel in the composition (e.g., a dosage or unit dosage form) is in the range of about 5 mg to about 500 mg, such as about 30 mg to about 300 mg or about 50 mg to about 200 mg, of the derivative. In some embodiments, the carrier is suitable for parental administration (e.g., intravenous administration). In some embodiments, the paclitaxel is the only pharmaceutically active agent for the treatment of cancer that is contained in the composition.

In some embodiments, there is provided a dosage form (e.g., a unit dosage form) for the treatment of cancer comprising any one of the compositions (such as pharmaceutical compositions) described herein. In some embodiments, there are provided articles of manufacture comprising the compositions, formulations, and unit dosages described herein in suitable packaging for use in the methods of treatment, methods of administration, and dosage regimes described herein. Suitable packaging for compositions described herein are known in the art, and include, for example, vials (such as sealed vials), vessels (such as sealed vessels), ampules, bottles, jars, flexible packaging (e.g., sealed Mylar or plastic bags), and the like. These articles of manufacture may further be sterilized and/or sealed.

In some embodiments, there is provided a commercial batch of a composition (such as pharmaceutical composition) described herein. “Commercial batch” used herein refers to a batch size that is at least about 20 grams (by weight of paclitaxel). In some embodiments, the batch size is at least about 30, 40, 50, 60, 70, 80, 90, 100, 150, 200, 250, 300, 350, 400, 450, 500, 550, 600, 650, 700, 750, 800, 850, 900, 1000, 1500, 2000, 2500, 3000, 3500, 4000, 4500, 5000, or 10,000 grams (by weight of paclitaxel).

Thus, the present application in some embodiments provides a commercial batch of a composition (such as pharmaceutical composition) comprising nanoparticles comprising albumin and paclitaxel, wherein no greater than about 2.4% of the total albumin in the composition is in the form of polymers. In some embodiments there is provided a commercial batch of a composition (such as pharmaceutical composition) comprising nanoparticles comprising paclitaxel coated with albumin, wherein no greater than about 2.4% of the total albumin in the composition is in the form of polymers. In some embodiments, there is provided a commercial batch of a composition (such as pharmaceutical composition) comprising nanoparticles comprising paclitaxel coated with albumin, wherein the average diameter of the nanoparticles in the composition is no greater than about 200 nanometers, wherein no greater than about 2.4% of the total albumin in the composition is in the form of polymers. In some embodiments, there is provided a commercial batch of a composition (such as pharmaceutical composition) comprising nanoparticles comprising paclitaxel coated with human albumin, wherein the average diameter of the nanoparticles in the composition is no greater than about 150 nanometers (such as about 130 nm), wherein no greater than about 2.4% of the total albumin in the composition is in the form of polymers. In some embodiments, no greater than about 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the total albumin in the composition is in the form of polymers. In some embodiments, about 0% of the total albumin in the composition is in the form of polymers. In some embodiments, about 0% of the total albumin in the composition is in the form of polymers. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a commercial batch of a composition (such as pharmaceutical composition) comprising nanoparticles comprising albumin and paclitaxel, wherein at least about 92% of the total albumin in the composition is in the form of monomers. In some embodiments there is provided a commercial batch of a composition (such as pharmaceutical composition) comprising nanoparticles comprising paclitaxel coated with albumin, wherein at least about 92% of the total albumin in the composition is in the form of monomers. In some embodiments, there is provided a commercial batch of a composition (such as pharmaceutical composition) comprising nanoparticles comprising paclitaxel coated with albumin, wherein the average diameter of the nanoparticles in the composition is no greater than about 200 nanometers, and wherein at least about 92% of the total albumin in the composition is in the form of monomers. In some embodiments, there is provided a commercial batch of a composition (such as pharmaceutical composition) comprising nanoparticles comprising paclitaxel coated with human albumin, wherein the average diameter of the nanoparticles in the composition is no greater than about 150 nanometers (such as 130 nm), and wherein at least about 92% of the total albumin in the composition is in the form of monomers. In some embodiments, at least about 93%, 94%, or 95% of the total albumin in the composition is in the form of monomers. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a commercial batch of a composition (such as pharmaceutical composition) comprising nanoparticles comprising albumin and paclitaxel, wherein the weight ratio of albumin monomers to albumin polymers in the composition is at least about 33:1. In some embodiments there is provided a commercial batch of a composition (such as pharmaceutical composition) comprising nanoparticles comprising paclitaxel coated with albumin, wherein the weight ratio of albumin monomers to albumin polymers in the composition is at least about 33:1. In some embodiments, there is provided a commercial batch of a composition (such as pharmaceutical composition) comprising nanoparticles comprising paclitaxel coated with albumin, wherein the average diameter of the nanoparticles in the composition is no greater than about 200 nanometers, wherein the weight ratio of albumin monomers to albumin polymers in the composition is at least about 33:1. In some embodiments, there is provided a commercial batch of a composition (such as pharmaceutical composition) comprising nanoparticles comprising paclitaxel coated with human albumin, wherein the average diameter of the nanoparticles in the composition is no greater than about 150 nanometers (such as about 130 nm), wherein the weight ratio of albumin monomers to albumin polymers in the composition is at least about 33:1. In some embodiments, the weight ratio of albumin monomers to albumin polymers in the composition is at least about any of 34:1, 35:1, 36:1, 37:1, 38:1, 39:1, 40:1, 41:1, 42:1, 43:1, 44:1, 45:1, 46:1, 47:1, or 48:1. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a commercial batch of a composition (such as pharmaceutical composition) comprising nanoparticles comprising albumin and paclitaxel, wherein at least about 80% of the total albumin in the composition is in the form of monomers, and wherein no greater than about 2.4% of the total albumin in the composition is in the form of polymers. In some embodiments there is provided a commercial batch of a composition (such as pharmaceutical composition) comprising nanoparticles comprising paclitaxel coated with albumin, wherein at least about 80% of the total albumin in the composition is in the form of monomers, and wherein no greater than about 2.4% of the total albumin in the composition is in the form of polymers. In some embodiments, there is provided a commercial batch of a composition (such as pharmaceutical composition) comprising nanoparticles comprising paclitaxel coated with albumin, wherein the average diameter of the nanoparticles in the composition is no greater than about 200 nanometers, wherein at least about 80% of the total albumin in the composition is in the form of monomers, and wherein no greater than about 2.4% of the total albumin in the composition is in the form of polymers. In some embodiments, there is provided a commercial batch of a composition (such as pharmaceutical composition) comprising nanoparticles comprising paclitaxel coated with human albumin, wherein the average diameter of the nanoparticles in the composition is no greater than about 150 nanometers (such as about 130 nm), wherein at least about 80% of the total albumin in the composition is in the form of monomers, and wherein no greater than about 2.4% of the total albumin in the composition is in the form of polymers. In some embodiments, at least about 81%, 82%, 83%, 84%, 85%, 86%, 87%, 88%, 89%, 90%, 91%, 92%, 93%, 94%, or 95% of the total albumin in the composition is in the form of monomers. In some embodiments, no greater than about 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the total albumin in the composition is in the form of polymers. In some embodiments, about 0% of the total albumin in the composition is in the form of polymers. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a commercial batch of a composition (such as pharmaceutical composition) comprising nanoparticles comprising albumin and paclitaxel, wherein no greater than about 2.4% of the total albumin in the composition is in the form of polymers, and wherein the weight ratio of albumin monomers to albumin polymers in the composition is at least about 33:1. In some embodiments there is provided a commercial batch of a composition (such as pharmaceutical composition) comprising nanoparticles comprising paclitaxel coated with albumin, wherein no greater than about 2.4% of the total albumin in the composition is in the form of polymers, and wherein the weight ratio of albumin monomers to albumin polymers in the composition is at least about 33:1. In some embodiments, there is provided a commercial batch of a composition (such as pharmaceutical composition) comprising nanoparticles comprising paclitaxel coated with albumin, wherein the average diameter of the nanoparticles in the composition is no greater than about 200 nanometers, wherein no greater than about 2.4% of the total albumin in the composition is in the form of polymers, and wherein the weight ratio of albumin monomers to albumin polymers in the composition is at least about 33:1. In some embodiments, there is provided a commercial batch of a composition (such as pharmaceutical composition) comprising nanoparticles comprising paclitaxel coated with human albumin, wherein the average diameter of the nanoparticles in the composition is no greater than about 150 nanometers (such as about 130 nm), wherein no greater than about 2.4% of the total albumin in the composition is in the form of polymers, and wherein the weight ratio of albumin monomers to albumin polymers in the composition is at least about 33:1. In some embodiments, no greater than about 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the total albumin in the composition is in the form of polymers. In some embodiments, about 0% of the total albumin in the composition is in the form of polymers. In some embodiments, the weight ratio of albumin monomers to albumin polymers in the composition is at least about any of 33:1, 34:1, 35:1, 36:1, 37:1, 38:1, 39:1, 40:1, 41:1, 42:1, 43:1, 44:1, 45:1, 46:1, 47:1, or 48:1. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, there is provided a commercial batch of a composition (such as pharmaceutical composition) comprising nanoparticles comprising albumin and paclitaxel, wherein at least about 80% of the total albumin in the composition is in the form of monomers, wherein no greater than about 2.4% of the total albumin in the composition is in the form of polymers, wherein no greater than about 15% (such as about 4% to about 15%, for example about 4% to about 10%) of the total albumin in the composition is in the form of dimers, and wherein no greater than about 10% (such as no greater than about 5%, for example no greater than about 1%) of the total albumin in the composition is in the form of oligomers. In some embodiments there is provided a commercial batch of a composition (such as pharmaceutical composition) comprising nanoparticles comprising paclitaxel coated with albumin, wherein at least about 80% of the total albumin in the composition is in the form of monomers, wherein no greater than about 2.4% of the total albumin in the composition is in the form of polymers, wherein no greater than about 15% (such as about 4% to about 15%, for example about 4% to about 10%) of the total albumin in the composition is in the form of dimers, and wherein no greater than about 10% (such as no greater than about 5%, for example no greater than about 1%) of the total albumin in the composition is in the form of oligomers. In some embodiments, there is provided a commercial batch of a composition (such as pharmaceutical composition) comprising nanoparticles comprising paclitaxel coated with albumin, wherein the average diameter of the nanoparticles in the composition is no greater than about 200 nanometers, wherein at least about 80% of the total albumin in the composition is in the form of monomers, wherein no greater than about 2.4% of the total albumin in the composition is in the form of polymers, wherein no greater than about 15% (such as about 4% to about 15%, for example about 4% to about 10%) of the total albumin in the composition is in the form of dimers, and wherein no greater than about 10% (such as no greater than about 5%, for example no greater than about 1%) of the total albumin in the composition is in the form of oligomers. In some embodiments, there is provided a commercial batch of a composition (such as pharmaceutical composition) comprising nanoparticles comprising paclitaxel coated with human albumin, wherein the average diameter of the nanoparticles in the composition is no greater than about 150 nanometers (such as about 130 nm), wherein at least about 80% of the total albumin in the composition is in the form of monomers, wherein no greater than about 2.4% of the total albumin in the composition is in the form of polymers, wherein no greater than about 15% (such as about 4% to about 15%, for example about 4% to about 10%) of the total albumin in the composition is in the form of dimers, and wherein no greater than about 10% (such as no greater than about 5%, for example no greater than about 1%) of the total albumin in the composition is in the form of oligomers. In some embodiments, at least about 60% of the monomeric albumins in the composition have a free thiol group. In some embodiments, at least about 60% of the monomeric albumins in the composition have a blocked thiol group. In some embodiments, the composition comprises albumin not associated with the nanoparticles. In some embodiments, the weight ratio of the albumin and the paclitaxel in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of the albumin and the paclitaxel in the composition is about 9:1.

In some embodiments, the commercial batch of the composition is substantially free of fatty acid, caprylate, and/or tryptophanate. In some embodiments, the commercial batch of the composition is substantially free of albumin lacking C-terminal Leu and/or albumin lacking N-terminal Asp-Ala. In some embodiments, the commercial batch of the composition (such as pharmaceutical composition) has an albumin glycosylation profile that is different from that of albumin obtained from natural sources (e.g., from human). The commercial batch of the composition (such as pharmaceutical composition) can have any one or more of the above characteristics. In some embodiments, the commercial batch of the composition (such as pharmaceutical composition) has none of the above characteristics. In some embodiments, the commercial batch of the composition (such as pharmaceutical composition) has all of the above characteristics.

Kits

The present application also provides kits comprising the compositions, formulations, unit dosages, and articles of manufacture described herein for use in the methods of treatment, methods of administration, and dosage regimes described herein. Kits described herein include one or more containers comprising the paclitaxel nanoparticle compositions (formulations or unit dosage forms and/or articles of manufacture), and in some embodiments, further comprise instructions for use in accordance with any of the methods of treatment described herein. In various embodiments, the amount of paclitaxel in the kit is included in any one of the following ranges: about 5 mg to about 20 mg, about 20 to about 50 mg, about 50 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of paclitaxel in the kit is in the range of about 5 mg to about 500 mg, such as about 30 mg to about 300 mg or about 50 mg to about 200 mg. In some embodiments, the kit includes one or more other compounds (e.g., one or more compounds other than paclitaxel that are useful for cancer).

Instructions supplied in the kits described herein are typically written instructions on a label or package insert (e.g., a paper sheet included in the kit), but machine-readable instructions (e.g., instructions carried on a magnetic or optical storage disk) are also acceptable. The instructions relating to the use of the nanoparticle compositions generally include information as to dosage, dosing schedule, and route of administration for the intended treatment. The kit may further comprise a description of selecting an individual suitable or treatment.

The present application also provides kits comprising compositions (or unit dosages forms and/or articles of manufacture) described herein and may further comprise instruction(s) on methods of using the composition, such as uses further described herein. In some embodiments, the kit described herein comprises the packaging described above. In other variations, the kit described herein comprises the packaging described above and a second packaging comprising a buffer. It may further include other materials desirable from a commercial and user standpoint, including other buffers, diluents, filters, needles, syringes, and package inserts with instructions for performing any methods described herein.

For combination therapies described herein, the kit may contain instructions for administering the first and second therapies simultaneously and/or sequentially for the effective treatment of cancer. The first and second therapies can be present in separate containers or in a single container. It is understood that the kit may comprise one distinct composition or two or more compositions wherein one composition comprises a first therapy and one composition comprises a second therapy.

Kits may also be provided that contain sufficient dosages of the paclitaxel as disclosed herein to provide effective treatment for an individual for an extended period, such as any one of a week, 2 weeks, 3 weeks, 4 weeks, 6 weeks, 8 weeks, 3 months, 4 months, 5 months, 6 months, 7 months, 8 months, 9 months or more. Kits may also include multiple unit doses of the paclitaxel, compositions (such as pharmaceutical compositions), and formulations described herein and instructions for use and packaged in quantities sufficient for storage and use in pharmacies, for example, hospital pharmacies and compounding pharmacies. In some embodiments, the kit comprises a dry (e.g., lyophilized) composition that can be reconstituted, resuspended, or rehydrated to form generally a stable aqueous suspension of nanoparticles comprising paclitaxel and albumin.

The kits described herein are in suitable packaging. Suitable packaging include, but is not limited to, vials, bottles, jars, flexible packaging (e.g., sealed Mylar or plastic bags), and the like. Kits may optionally provide additional components such as buffers and interpretative information.

### Methods of Making the Nanoparticle Compositions

The present application also provides methods of making the paclitaxel nanoparticle compositions described herein. Nanoparticles containing poorly water soluble pharmaceutical agents and carrier proteins (e.g., albumin) can be prepared under conditions of high shear forces (e.g., sonication, high pressure homogenization, or the like). These methods are disclosed in, for example, U.S. Pat. Nos. 5,916,596; 6,096,331; 6,749,868; 6,537,579; and PCT Application Pub. Nos. WO98/14174; WO99/00113; WO07/027941; and WO07/027819. The contents of these publications, particularly with respect the method of making composition containing carrier proteins, are hereby incorporated by reference in their entireties.

Generally, to make the paclitaxel nanoparticle compositions described herein, paclitaxel is dissolved in an organic solvent. Suitable organic solvents include, for example, ketones, esters, ethers, chlorinated solvents, and other solvents known in the art. For example, the organic solvent can be methylene chloride/ethanol, chloroform/ethanol, or chloroform/t-butanol (for example with a ratio of about any one of 1:9, 1:8, 1:7, 1:6, 1:5, 1:4, 1:3, 1:2, 1:1, 2:1, 3:1, 4:1, 5:1, 6:1, 7:1, 8:1, or 9:1 or with a ratio of about any one of 3:7, 5:7, 4:6, 5:5, 6:5, 8:5, 9:5, 9.5:5, 5:3, 7:3, 6:4, or 9.5:0.5). Albumin (such as recombinant albumin, for example Novozyme recombinant albumin or Intrivia recombinant albumin disclosed herein) is dissolved in water and combined with the paclitaxel solution. The mixture is subjected to high pressure homogenization (e.g., using an Avestin, APV Gaulin, Microfluidizer™ such as a Microfluidizer™ Processor M-110EH from Microfluidics, Stansted, or Ultra Turrax homogenizer). The emulsion may be cycled through the high pressure homogenizer for between about 2 to about 100 cycles, such as about 5 to about 50 cycles or about 8 to about 20 cycles (e.g., about any one of 8, 10, 12, 14, 16, 18 or 20 cycles). The organic solvent can then be removed by evaporation utilizing suitable equipment known for this purpose, including, but not limited to, rotary evaporators, falling film evaporators, wiped film evaporators, spray driers, and the like that can be operated in batch mode or in continuous operation. The solvent may be removed at reduced pressure (such as at about any one of 25 mm Hg, 30 mm Hg, 40 mm Hg, 50 mm Hg, 100 mm Hg, 200 mm Hg, or 300 mm Hg). The amount of time used to remove the solvent under reduced pressure may be adjusted based on the volume of the formulation. For example, for a formulation produced on a 300 mL scale, the solvent can be removed at about 1 to about 300 mm Hg (e.g., about any one of 5-100 mm Hg, 10-50 mm Hg, 20-40 mm Hg, or 25 mm Hg) for about 5 to about 60 minutes (e.g., about any one of 7, 8, 9, 10, 11, 12, 13, 14, 15 16, 18, 20, 25, or 30 minutes). The dispersion obtained can be further lyophilized.

If desired, additional albumin solution may be added to the dispersion to adjust the albumin to paclitaxel ratio, or to adjust the concentration of paclitaxel in the dispersion. For example, albumin solution (e.g., 25% w/v) can be added to adjust the albumin to paclitaxel ratio to about any one of 18:1, 15:1 14:1, 13:1, 12:1, 11:1, 10:1, 9:1, 8:1, 7.5:1, 7:1, 6:1, 5:1, 4:1, or 3:1. In another example, albumin solution (e.g., 25% w/v) or another solution is added to adjust the concentration of paclitaxel in the dispersion to about any one of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml. The dispersion may be serially filtered through multiple filters, such as a combination of 1.2 μm and 0.8/0.2 μm filters; the combination of 1.2 μm, 0.8 μm, 0.45 μm, and 0.22 μm filters; or the combination of any other filters known in the art. The dispersion obtained can be further lyophilized. The nanoparticle compositions may be made using a batch process or a continuous process (e.g., the production of a composition on a large scale).

If desired, a second therapy (e.g., one or more compounds useful for treating cancer), an antimicrobial agent, sugar, and/or stabilizing agent can also be included in the composition. For example, this additional agent can either be admixed with paclitaxel and/or the albumin during the preparation of the paclitaxel nanoparticle composition, or added after the paclitaxel nanoparticle composition is prepared. In some embodiments, the agent is admixed with the paclitaxel nanoparticle composition prior to lyophilization. In some embodiments, the agent is added to the lyophilized paclitaxel nanoparticle composition. In some embodiments when the addition of the agent changes the pH of the composition, the pH in the composition are generally (but not necessarily) adjusted to a desired pH. Exemplary pH values of the compositions include, for example, in the range of about 5 to about 8.5. In some embodiments, the pH of the composition is adjusted to no less than about 6, including for example no less than any one of about 6.5, 7, or 8 (e.g., about 8).

### Methods of Treating Diseases

The nanoparticle compositions of the present invention may be used to treat diseases associated with cellular proliferation or hyperproliferation, such as cancers.

Examples of cancers that may be treated by the methods described herein include, but are not limited to, breast cancer (such as metastatic breast cancer), lung cancer (such as non-small cell lung cancer), pancreatic cancer (such as metastatic pancreatic cancer or locally advanced unresectable pancreatic cancer), multiple myeloma, renal cell carcinoma, prostate cancer, melanoma (such as metastatic melanoma), colon cancer, colorectal cancer, ovarian cancer, liver, renal, and gastric cancer. In some embodiments, the cancer is breast cancer after failure of combination chemotherapy for metastatic disease or relapse within 6 months of adjuvant chemotherapy. In some embodiments, the prior therapy includes an anthracycline treatment.

Cancers to be treated by compositions described herein include, but are not limited to, carcinoma, lymphoma, blastoma, sarcoma, and leukemia. Examples of cancers that can be treated by compositions described herein include, but are not limited to, squamous cell cancer, lung cancer (including small cell lung cancer, non-small cell lung cancer, adenocarcinoma of the lung, and squamous carcinoma of the lung, including squamous NSCLC), cancer of the peritoneum, hepatocellular cancer, gastric or stomach cancer (including gastrointestinal cancer), pancreatic cancer (such as advanced pancreatic cancer), glioblastoma, cervical cancer, ovarian cancer, liver cancer (such as hepatocellular carcinoma), bladder cancer, hepatoma, breast cancer, colon cancer, melanoma, endometrial or uterine carcinoma, salivary gland carcinoma, kidney or renal cancer, liver cancer, prostate cancer (such as advanced prostate cancer), vulval cancer, thyroid cancer, hepatic carcinoma, head and neck cancer, colorectal cancer, rectal cancer, soft-tissue sarcoma, Kaposi's sarcoma, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's macroglobulinemia), chronic lymphocytic leukemia (CLL), acute lymphoblastic leukemia (ALL), myeloma, Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome. In some embodiments, there is provided a method of treating metastatic cancer (that is, cancer that has metastasized from the primary tumor). In some embodiments, there is provided a method of reducing cell proliferation and/or cell migration. In some embodiments, there is provided a method of treating hyperplasia, for example hyperplasia in the vascular system that can result in restenosis or hyperplasia that can result in arterial or venous hypertension.

In some embodiments, there are provided methods of treating cancer at advanced stage(s). In some embodiments, there are provided methods of treating breast cancer (which may be HER2 positive or HER2 negative), including, for example, advanced breast cancer, stage IV breast cancer, locally advanced breast cancer, and metastatic breast cancer. In some embodiments, the cancer is lung cancer, including, for example, non-small cell lung cancer (NSCLC, such as advanced NSCLC), small cell lung cancer (SCLC, such as advanced SCLC), and advanced solid tumor malignancy in the lung. In some embodiments, the cancer is ovarian cancer, head and neck cancer, gastric malignancies, melanoma (including metastatic melanoma), colorectal cancer, pancreatic cancer, and solid tumors (such as advanced solid tumors). In some embodiments, the cancer is any of (and in some embodiments selected from the group consisting of) breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, gliomas, glioblastomas, neuroblastomas, and multiple myeloma. In some embodiments, the cancer is a solid tumor.

In some embodiments, the cancer to be treated is breast cancer, such as metastatic breast cancer. In some embodiments, the cancer to be treated is lung cancer, such as non-small cell lung cancer, including advanced stage non-small cell lung cancer. In some embodiments, the cancer to be treated is pancreatic cancer, such as early stage pancreatic cancer or advanced or metastatic pancreatic cancer. In some embodiments, the cancer to be treated is melanoma, such as stage III or IV melanoma.

In some embodiments, the individual being treated for a proliferative disease has been identified as having one or more of the conditions described herein. Identification of the conditions as described herein by a skilled physician is routine in the art (e.g., via blood tests, X-rays, CT scans, endoscopy, biopsy, angiography, CT-angiography, etc.) and may also be suspected by the individual or others, for example, due to tumor growth, hemorrhage, ulceration, pain, enlarged lymph nodes, cough, jaundice, swelling, weight loss, cachexia, sweating, anemia, paraneoplastic phenomena, thrombosis, etc. In some embodiments, the individual has been identified as susceptible to one or more of the conditions as described herein. The susceptibility of an individual may be based on any one or more of a number of risk factors and/or diagnostic approaches appreciated by the skilled artisan, including, but not limited to, genetic profiling, family history, medical history (e.g., appearance of related conditions), lifestyle or habits.

In some embodiments, the methods and/or compositions used herein reduce the severity of one or more symptoms associated with proliferative disease (e.g., cancer) by at least about any one of 10%, 20%, 30%, 40%, 50%, 60%, 70%, 80%, 90%, 95%, or 100% compared to the corresponding symptom in the same individual prior to treatment or compared to the corresponding symptom in other individuals not receiving the methods and/or compositions.

In some embodiments, the composition (such as pharmaceutical composition) described herein is used in combination with another administration modality or treatment.

### Dosing and Method of Administration

The amount of the pharmaceutical composition administered to an individual (such as a human) may vary with the particular composition, the method of administration, and the particular type of recurrent cancer being treated. The amount should be sufficient to produce a desirable beneficial effect. For example, in some embodiments, the amount of the composition is effective to result in an objective response (such as a partial response or a complete response). In some embodiments, the amount of nanoparticle composition is sufficient to result in a complete response in the individual. In some embodiments, the amount of the composition is sufficient to result in a partial response in the individual. In some embodiments, the amount of the composition administered alone is sufficient to produce an overall response rate of more than about any one of 40%, 50%, 60%, or 64% among a population of individuals treated with the composition. Responses of an individual to the treatment of the methods described herein can be determined, for example, based on RECIST or CA-125 level. For example, when CA-125 is used, a complete response can be defined as a return to a normal range value of at least 28 days from the pretreatment value. A particle response can be defined as a sustained over 50% reduction from the pretreatment value.

In some embodiments, the amount of nanoparticle composition is sufficient to prolong progress-free survival of the individual (for example as measured by RECIST or CA-125 changes). In some embodiments, the amount of the nanoparticle composition is sufficient to prolong overall survival of the individual. In some embodiments, the amount of the composition is sufficient to produce clinical benefit of more than about any one of 50%, 60%, 70%, or 77% among a population of individuals treated with the composition.

In some embodiments, the amount of paclitaxel in the composition is below the level that induces a toxicological effect (i.e., an effect above a clinically acceptable level of toxicity) or is at a level where a potential side effect can be controlled or tolerated when the composition is administered to the individual. In some embodiments, the amount of the composition is close to a maximum tolerated dose (MTD) of the composition following the same dosing regime. In some embodiments, the amount of the composition is more than about any one of 80%, 90%, 95%, or 98% of the MTD.

In some embodiments, the amount of paclitaxel and/or composition is an amount sufficient to decrease the size of a tumor, decrease the number of cancer cells, or decrease the growth rate of a tumor by at least about any one of 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of paclitaxel in the composition is included in any one of the following ranges: about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of paclitaxel in the composition (e.g., a unit dosage form) is in the range of about 5 mg to about 500 mg, such as about 30 mg to about 300 mg or about 50 mg to about 200 mg. In some embodiments, the concentration of the paclitaxel in the composition is dilute (about 0.1 mg/ml) or concentrated (about 100 mg/ml), including for example any one of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml. In some embodiments, the concentration of the paclitaxel is at least about any one of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml.

Exemplary doses of paclitaxel in the nanoparticle composition include, but are not limited to, about any one of 25 mg/m², 30 mg/m², 50 mg/m², 60 mg/m², 75 mg/m², 80 mg/m², 90 mg/m², 100 mg/m², 120 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² of paclitaxel. In various embodiments, the composition includes less than about any one of 350 mg/m², 300 mg/m², 250 mg/m², 200 mg/m², 150 mg/m², 120 mg/m², 100 mg/m², 90 mg/m², 50 mg/m², or 30 mg/m² of paclitaxel. In some embodiments, the amount of paclitaxel per administration is less than about any one of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the dose of paclitaxel in the composition is included in any one of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². Preferably, the dose of paclitaxel in the composition is about 5 to about 300 mg/m², such as about 100 to about 150 mg/m², about 120 mg/m², about 130 mg/m², or about 140 mg/m². In some embodiments, the nanoparticles comprising paclitaxel are not administered at a dose of 300 mg/m² or 900 mg/m².

In some embodiments of any of the above aspects, the dose of paclitaxel in the composition includes at least about any one of 1 mg/kg, 2.5 mg/kg, 3.5 mg/kg, 5 mg/kg, 6.5 mg/kg, 7.5 mg/kg, 10 mg/kg, 15 mg/kg, or 20 mg/kg. In various variations, the dose of paclitaxel in the composition includes less than about any one of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, 2 mg/kg, 1.5 mg/kg, or 1 mg/kg of paclitaxel. In some embodiments, the dose of paclitaxel in the composition includes less than about any one of 500 μg/kg, 350 μg/kg, 300 μg/kg, 250 μg/kg, 200 μg/kg, 150 μg/kg, 100 μg/kg, 50 μg/kg, 25 μg/kg, 20 μg/kg, 10 μg/kg, 7.5 μg/kg, 6.5 μg/kg, 5 μg/kg, 3.5 μg/kg, 2.5 μg/kg, 2 μg/kg, 1.5 μg/kg, 1 μg/kg, or 0.5 μg/kg of paclitaxel. In some embodiments, the nanoparticles comprising paclitaxel are not administered at a dose of 60 mg/kg or 90 mg/kg.

Exemplary dosing frequencies include, but are not limited to, any one of weekly without break; weekly, three out of four weeks; once every three weeks; once every two weeks; weekly, two out of three weeks. In some embodiments, the composition is administered about once every 2 weeks, once every 3 weeks, once every 4 weeks, once every 6 weeks, or once every 8 weeks. In some embodiments, the composition is administered at least about any one of 1×, 2×, 3×, 4×, 5×, 6×, or 7× (i.e., daily) a week. In some embodiments, the intervals between each administration are less than about any one of 6 months, 3 months, 1 month, 20 days, 15, days, 12 days, 10 days, 9 days, 8 days, 7 days, 6 days, 5 days, 4 days, 3 days, 2 days, or 1 day. In some embodiments, the intervals between each administration are more than about any one of 1 month, 2 months, 3 months, 4 months, 5 months, 6 months, 8 months, or 12 months. In some embodiments, there is no break in the dosing schedule. In some embodiments, the interval between each administration is no more than about a week.

The administration of the composition can be extended over an extended period of time, such as from about a month up to about seven years. In some embodiments, the composition is administered over a period of at least about any one of 2, 3, 4, 5, 6, 7, 8, 9, 10, 11, 12, 18, 24, 30, 36, 48, 60, 72, or 84 months. In some embodiments, the composition is administered over a period of at least one month, wherein the interval between each administration is no more than about a week, and wherein the dose of paclitaxel at each administration is about 0.25 mg/m² to about 75 mg/m², such as about 0.25 mg/m² to about 25 mg/m² or about 25 mg/m² to about 50 mg/m².

In some embodiments, the dosage of paclitaxel in a nanoparticle composition can be in the range of 5-400 mg/m² when given on a 3 week schedule, or 5-250 mg/m² when given on a weekly schedule. For example, the amount of a paclitaxel is about 60 to about 300 mg/m² (e.g., about 260 mg/m²).

Other exemplary dosing schedules for the administration of the nanoparticle composition include, but are not limited to, any one of 100 mg/m², weekly, without break; 75 mg/m² weekly, 3 out of four weeks; 100 mg/m², weekly, 3 out of 4 weeks; 125 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², weekly, without break; 20-150 mg/m² twice a week; and 150-250 mg/m² twice a week. The dosing frequency of the composition may be adjusted over the course of the treatment based on the judgment of the administering physician.

In some embodiments, the composition is administered (e.g., intravenously) at 260 mg/m2 every three weeks. In some embodiments, the composition is administered (e.g., intravenously) at 220 mg/m², every three weeks. In some embodiments, the composition is administered (e.g., intravenously) at 180 mg/m², every three weeks. In some embodiments, the composition is administered (e.g., intravenously) at 200 mg/m², every three weeks. In some embodiments, the composition is administered (e.g., intravenously) at 130 mg/m², every three weeks.

In some embodiments, the composition is administered (e.g., intravenously) at 150 mg/m² on days 1, 8, and 15 every 4 weeks. In some embodiments, the composition is administered (e.g., intravenously) at 125 mg/m2 on days 1, 8, and 15 every 4 weeks. In some embodiments, the composition is administered (e.g., intravenously) at 100 mg/m² on days 1, 8, and 15 every 4 weeks. In some embodiments, the composition is administered (e.g., intravenously) at 75 mg/m2 on days 1, 8, and 15 every 4 weeks. In some embodiments, the composition is administered (e.g., intravenously) at 50 mg/m² on days 1, 8, and 15 every 4 weeks.

The compositions described herein allow infusion of the composition to an individual over an infusion time that is shorter than about 24 hours. For example, in some embodiments, the composition is administered over an infusion period of less than about any one of 24 hours, 12 hours, 8 hours, 5 hours, 3 hours, 2 hours, 1 hour, 30 minutes, 20 minutes, or 10 minutes. In some embodiments, the composition is administered over an infusion period of about 30 minutes. In some embodiments, the composition is administered over an infusion period between about 30 minutes to about 40 minutes.

In some embodiments, the present application provides a method of treating cancer in an individual by parenterally administering to the individual (e.g., a human) an effective amount of a composition (such as pharmaceutical composition) described herein. The present application also provides a method of treating cancer in an individual by intravenous, intra-arterial, intramuscular, subcutaneous, inhalation, oral, intraperitoneal, nasally, or intra-tracheal administering to the individual (e.g., a human) an effective amount of a paclitaxel nanoparticle composition. In some embodiments, the route of administration is intraperitoneal. In some embodiments, the route of administration is intravenous, intra-arterial, intramuscular, or subcutaneous. In various variations, about 5 mg to about 500 mg, such as about 30 mg to about 300 mg or about 50 to about 500 mg, of the paclitaxel is administered per dose. In some embodiments, the paclitaxel is the only pharmaceutically active agent for the treatment of cancer that is contained in the composition.

Any of the compositions described herein can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, transdermal, intratumoral, direct injection into the blood vessel wall, intracranial, or intra-cavity. In some embodiments, sustained continuous release formulation of the composition may be used. In one variation described herein, nanoparticles (such as albumin nanoparticles) of the inventive compounds can be administered by any acceptable route including, but not limited to, orally, intramuscularly, transdermally, intravenously, through an inhaler or other air borne delivery systems and the like.

In some embodiments, drug-containing nanoparticle compositions may be administered with a second therapeutic compound and/or a second therapy. The dosing frequency of the composition and the second compound may be adjusted over the course of the treatment based on the judgment of the administering physician. In some embodiments, the first and second therapies are administered simultaneously, sequentially, or concurrently. When administered separately, the nanoparticle composition and the second compound can be administered at different dosing frequency or intervals. For example, the composition can be administered weekly, while a second compound can be administered more or less frequently. In some embodiments, sustained continuous release formulation of paclitaxel-containing nanoparticle and/or second compound may be used. Various formulations and devices for achieving sustained release are known in the art. A combination of the administration configurations described herein can be used.

### Metronomic Therapy Regimes

The present invention also provides metronomic therapy regimes for any of the methods of treatment and methods of administration described herein. Exemplary metronomic therapy regimes and variations for the use of metronomic therapy regimes are discussed below and disclosed in U.S. Ser. No. 11/359,286, filed Feb. 21, 2006, published as U.S. Pub. No. 2006/0263434 (such as those described in paragraphs [0138] to [0157] therein), which is hereby incorporated by reference in its entirety. In some embodiments, the nanoparticle composition is administered over a period of at least one month, wherein the interval between each administration is no more than about a week, and wherein the dose of the paclitaxel at each administration is about 0.25% to about 25% of its maximum tolerated dose following a traditional dosing regime. In some embodiments, the nanoparticle composition is administered over a period of at least two months, wherein the interval between each administration is no more than about a week, and wherein the dose of the paclitaxel at each administration is about 1% to about 20% of its maximum tolerated dose following a traditional dosing regime. In some embodiments, the dose of paclitaxel per administration is less than about any one of 25%, 24%, 23%, 22%, 20%, 18%, 15%, 14%, 13%, 12%, 11%, 10%, 9%, 8%, 7%, 6%, 5%, 4%, 3%, 2%, or 1% of the maximum tolerated dose. In some embodiments, any nanoparticle composition is administered at least about any one of 1×, 2×, 3×, 4×, 5×, 6×, or 7× (i.e., daily) a week. In some embodiments, the intervals between each administration are less than about any one of 6 months, 3 months, 1 month, 20 days, 15, days, 12 days, 10 days, 9 days, 8 days, 7 days, 6 days, 5 days, 4 days, 3 days, 2 days, or 1 day. In some embodiments, the intervals between each administration are more than about any one of 1 month, 2 months, 3 months, 4 months, 5 months, 6 months, 8 months, or 12 months. In some embodiments, the composition is administered over a period of at least about any one of 2, 3, 4, 5, 6, 7, 8, 9, 10, 11, 12, 18, 24, 30, 36, 48, 60, 72, or 84 months.

### Exemplary Embodiments

The present application in some embodiments provides a composition (such as pharmaceutical composition) comprising nanoparticles comprising albumin and paclitaxel, wherein no greater than about 2.4% of the total albumin in the composition (such as pharmaceutical composition) is in the form of polymers.

In some embodiments according to (or as applied to) any of the embodiments above, at least about 80% of the total albumin in the composition (such as pharmaceutical composition) is in the form of monomers.

In some embodiments according to (or as applied to) any of the embodiments above, at least about 92% of the total albumin in the composition (such as pharmaceutical composition) is in the form of monomers.

In some embodiments according to (or as applied to) any of the embodiments above, at least about 60% of the monomeric albumins in the composition (such as pharmaceutical composition) have a free thiol group.

In some embodiments according to (or as applied to) any of the embodiments above, at least about 60% of the monomeric albumin in the composition (such as pharmaceutical composition) have a blocked thiol group.

In some embodiments according to (or as applied to) any of the embodiments above, no greater than about 10% of total albumin in the composition (such as pharmaceutical composition) is in the form of dimers.

In some embodiments according to (or as applied to) any of the embodiments above, no greater than about 3% of total albumin in the composition (such as pharmaceutical composition) is in the form of oligomers.

In some embodiments according to (or as applied to) any of the embodiments above, the composition (such as pharmaceutical composition) is substantially free of albumin lacking C-terminal Leu and albumin lacking N-terminal Asp-Ala.

In some embodiments according to (or as applied to) any of the embodiments above, the albumin in the composition (such as pharmaceutical composition) has a glycosylation profile that is different from that of native albumin obtained from a human.

In some embodiments according to (or as applied to) any of the embodiments above, the albumin in the composition (such as pharmaceutical composition) has no glycosylation.

In some embodiments according to (or as applied to) any of the embodiments above, the composition (such as pharmaceutical composition) is substantially free of fatty acids.

In some embodiments according to (or as applied to) any of the embodiments above, the composition (such as pharmaceutical composition) is substantially free of caprylate.

In some embodiments according to (or as applied to) any of the embodiments above, the composition (such as pharmaceutical composition) is substantially free of tryptophan.

In some embodiments according to (or as applied to) any of the embodiments above, the composition (such as pharmaceutical composition) is substantially free of a blood component.

In some embodiments according to (or as applied to) any of the embodiments above, the composition (such as pharmaceutical composition) is substantially free of virus and prion.

In some embodiments according to (or as applied to) any of the embodiments above, no greater than about 0.5% of 7-epipaclitaxel is generated upon storage of the composition (such as pharmaceutical composition) at 55° C. for about two weeks.

In some embodiments according to (or as applied to) any of the embodiments above, no greater than about 0.7% of 7-epipaclitaxel is generated upon storage of the composition (such as pharmaceutical composition) at 55° C. for about 1 month.

In some embodiments according to (or as applied to) any of the embodiments above, no greater than about 0.45% total impurities were generated upon storage of the composition (such as pharmaceutical composition) at 55° C. for about two weeks.

In some embodiments according to (or as applied to) any of the embodiments above, no greater than about 0.65% total impurities were generated upon storage of the composition (such as pharmaceutical composition) at 55° C. for about 1 month.

In some embodiments according to (or as applied to) any of the embodiments above, no greater than about 1% additional albumin polymers are generated upon storage of the composition (such as pharmaceutical composition) at 55° C. for about two weeks.

In some embodiments according to (or as applied to) any of the embodiments above, no greater than about 1% additional albumin polymers are generated upon storage of the composition (such as pharmaceutical composition) at 55° C. for about 1 month.

In some embodiments according to (or as applied to) any of the embodiments above, no greater than about 10% albumin monomers are lost upon storage of the composition (such as pharmaceutical composition) at 55° C. for about two weeks.

In some embodiments according to (or as applied to) any of the embodiments above, no greater than about 20% albumin monomers are lost upon storage of the composition (such as pharmaceutical composition) at 55° C. for about 1 month.

In some embodiments according to (or as applied to) any of the embodiments above, at least about 80% of the total albumin in the composition (such as pharmaceutical composition) is not associated with the nanoparticles.

In some embodiments according to (or as applied to) any of the embodiments above, the nanoparticles comprise paclitaxel coated with albumin.

In some embodiments according to (or as applied to) any of the embodiments above, the nanoparticles in the composition (such as pharmaceutical composition) are substantially free of polymeric core matrix.

In some embodiments according to (or as applied to) any of the embodiments above, the nanoparticles in the composition (such as pharmaceutical composition) have an average diameter of no greater than about 200 nm.

In some embodiments according to (or as applied to) any of the embodiments above, the weight ratio of the albumin and the paclitaxel in the composition (such as pharmaceutical composition) is about 9:1 to about 1:1.

In some embodiments according to (or as applied to) any of the embodiments above, the weight ratio of the albumin and paclitaxel in the composition (such as pharmaceutical composition) is about 8:1 to about 1:1.

In some embodiments according to (or as applied to) any of the embodiments above, the composition (such as pharmaceutical composition) further comprises a sucrose.

In some embodiments according to (or as applied to) any of the embodiments above, the composition (such as pharmaceutical composition) does not comprise a sucrose.

In some embodiments according to (or as applied to) any of the embodiments above, the composition (such as pharmaceutical composition) further comprises an edetate.

In some embodiments according to (or as applied to) any of the embodiments above, the composition (such as pharmaceutical composition) does not comprises an edetate.

In some embodiments according to (or as applied to) any of the embodiments above, the albumin is human albumin.

The present application in some embodiments provides a commercial batch of a composition (such as pharmaceutical composition) according to (or as applied to) any of the embodiments above.

The present application in some embodiments provides method of treating a disease in an individual, comprising administering to the individual an effective amount of a composition (such as pharmaceutical composition) according to (or as applied to) any of the embodiments above.

In some embodiments according to (or as applied to) any of the embodiments above, the disease is cancer.

In some embodiments according to (or as applied to) any of the embodiments above, the individual is human.

### EXAMPLES

### Example 1

This example shows the preparation of paclitaxel/albumin nanoparticle compositions using recombinant human albumin (rHA) from Novozyme and human serum albumin from Baxter (HSA).

To make paclitaxel/albumin nanoparticles, 1.6 ml of organic phase (90:10 CHCl3/EtOH (v/v)) containing 200 mg/ml paclitaxel was added to 28.4 ml aqueous phase of albumin (52 mg/ml). The mixture was pre-homogenized using Silverson for 5 minutes at 5500 rpm, and then transferred into a high pressure homogenizer (Avestin). The homogenization was performed at 18,000-20,000 psi while recycling the emulsion for 12 passes. The resulting system was transferred into a Rotary evaporator, and organic solvents were removed at reduced pressure (40 mm of Hg), for 10-15 minutes. The resulting suspension was analyzed to determine the albumin content, particle size, and 72 hour hold-time. The suspension was then filtered through a sterile filters, filled into 10 ml vial (3 ml/vial), and lyophilized. Albumin content, albumin distribution, and particle size were analyzed before and after the filtration, and after lyophilization.

Formulations made with rHA and HSA had similar unfiltered and filtered particle sizes of about 140 nm. Both formulations were stable over 72 hours during an in-process hold. Both formulations filtered acceptably, and reconstitute to the same particle size. The reconstituted suspensions of both formulations were stable.

We observed, however, a difference in the albumin polymer/oligomer/monomer profile between formulations made with rHA and HSA. The albumin profile of the nanoparticle compositions are analyzed on size-exclusion chromatography. The conditions for the size-exclusion HPLC are set forth below:

A. Column: TOSOH TSKgel G3000 SWXL, 7.8×300 mm, 5 μm or equivalent
B. Guard Column: TOSOH TSKgel Guard SWXL, 6.0×40 mm, 7 μm, or equivalent

### C. Autosampler Temperature: Ambient

### D. Column Temperature: Ambient

### E. Detector Wavelength: 280 nm

F. Flow Rate: 1.0 mL/min

### G. Injection Volume: 50 μL

### H. Needle Wash Solvent: Water

I. Run Time: 22 minutes

The results of the analysis were summarized in Table 1.

TABLE 1

| Albumin | Batch | Monomer, % | Avg, % | Dimer, % | Avg, % | Oligomer, % | Avg, % | Polymer, % | Avg, % |
|---|---|---|---|---|---|---|---|---|---|
| HSA | 239- | 89.51 | 89.65 ± 0.25 | 6.05 | 5.97 ± 1.89 | 0.76 | 0.67 ± 0.15 | 3.68 | 3.71 ± 0.04 |
| | 239- | 89.49 | | 6.05 | | 0.75 | | 3.71 | |
| | 239- | 89.94 | | 5.82 | | 0.49 | | 3.75 | |
| rHA | 239- | 93.01 | 94.39 ± 1.89 | 6.24 | 5.92 ± 0.28 | 0.75 | 0.69 ± 0.06 | 0.00 | 0 |
| | 239- | 93.62 | | 5.73 | | 0.64 | | 0.00 | |
| | 239- | 96.55 | | 5.78 | | 0.68 | | 0.00 | |

### Example 2

This example shows the preparation of paclitaxel/albumin compositions using recombinant human albumin from Intrivia (rHSA) and human serum albumin from Baxter (HSA).

To make paclitaxel/albumin nanoparticles, 1.6 ml of organic phase (90:10 CHCl3/EtOH (v/v)) was added to 28.4 ml aqueous phase of albumin (52 mg/ml). The mixture was pre-homogenized using Silverson for 5 minutes at 5500 rpm, and then transferred into a high pressure homogenizer (Avestin). The homogenization was performed at 18,000-20,000 psi while recycling the emulsion for 12 passes. The resulting system was transferred into a Rotary evaporator, and organic solvents were rapidly removed at 40° C., at reduced pressure (40 mm of Hg), for 10-15 minutes. The resulting suspension was analyzed to determine the albumin content, particle size, and 72 hour hold-time. The suspension was then filtered through 1.2, 0.8, 0.45, and 0.22 μm syringe filters, filled into 10 ml vial (3 ml/vial), and lyophilized. Albumin content, albumin distribution, and particle size were analyzed before and after the filtration, and after lyophilization.

Formulations made with HSA had unfiltered particle size of 156 nm whereas the comparable formulation made with rHSA had unfiltered particle size of 173 nm. Both formulations exhibited stable particle sizes over 72 hours during an in-process hold. Both formulations filtered comparably. The recovery of paclitaxel of filtration was about 70%, for both. Both formulations reconstituted to the same particle size prior to lyophilization, and the reconstituted suspensions of both formulations were stable.

We observed a difference in the albumin polymer/oligomer/monomer profile between formulations made with rHA and HSA. The size-exclusion chromatography methods were carried out as shown in Example 1. The results of the analysis were summarized in Table 2.

TABLE 2

| Albumin | Batch # | Monomer, % | Avg, % | Dimer, % | Avg, % | Oligomer, % | Avg, % | Polymer, % | Avg, % |
|---|---|---|---|---|---|---|---|---|---|
| HSA | 1 | 91.87 | 91.76 ± 0.10 | 4.75 | 4.89 ± 0.12 | 0.00 | 0.00 | 3.38 | 3.35 ± 0.03 |
| | 3 | 91.70 | | 4.96 | | 0.00 | | 3.34 | |
| | 5 | 91.70 | | 4.97 | | 0.00 | | 3.32 | |
| rHSA | 2 | 93.93 | 94.02 ± 0.08 | 6.07 | 5.97 ± 0.09 | 0.00 | 0.00 | 0.00 | 0.00 |
| | 4 | 94.04 | | 5.96 | | 0.00 | | 0.00 | |
| | 6 | 94.09 | | 5.89 | | 0.00 | | 0.00 | |

### Example 3

This example further shows the analysis of different nanoparticle formulations. The different formulations used in this example are provided in Table 3. rHA refers to recombinant albumin obtained from Novozyme. HA refers to human serum albumin from Grifols. These formulations were prepared using the same high pressure homogenization methods described in Examples 1 and 2.

TABLE 3
NAB-PACLITAXEL formulations

| | NAB- | NAB- | NAB- | NAB- |
|---|---|---|---|---|
| Target | PACLITAXEL | PACLITAXEL | PACLITAXEL- | PACLITAXEL- |
| Composition | (rHA) | (HA) | NFZ | NFI |
| Paclitaxel | 100 mg/vial | | | |
| Human Albumin | 800 mg/vial | | | |
| (HA) | | | | |
| | Recombinant | | Recombinant | |
| | HA | HA | HA | HA |

| | | | |
|---|---|---|---|
| Sucrose | n/a | n/a | 450 mg/vial |
| EDTA | n/a | n/a | 1 mg/vial |

Albumin monomer/polymer profiles of the different paclitaxel/albumin nanoparticle formulations were analyzed using size-exclusion chromatography (HPLC). The conditions for the size-exclusion HPLC are set forth below:

A. Column (Guard): TOSOH BioScience, LLC Guard SWxL, 6.0 mm×40 mm, 7 μm
B. Column Temperature: ambient
C. Column: TOSOH BioScience, LLC TSKgel G3000SWxL, 7.8 mm×300 mm, 5 μm

### D. Detector Wavelength: 228 nm

E. Flow Rate: 1.0 mL/min

### F. Injection Volume 10 μL

G. Needle Wash: water
H. Run Time: 60 min (20 min or less for Standard Preparation 1, 2 and 3, and Confirmation Standard Preparation if no interference is found in the baseline)

Table 4 summarizes the albumin profiles.

TABLE 4
Comparison of albumin isomers for NAB-PACLITAXEL formulations
containing recombinant human albumin or human albumin.

| | Albumin Isomers (% of Solution) | | | |
|---|---|---|---|---|
| Formulations | Monomer | Dimer | Oligomer | Polymer |
| NAB- | 87 | 9 | 4 | 0 |
| PACLITAXEL | | | | |
| (rHA) | | | | |
| NAB- | 85 | 8 | 2 | 4.70 |
| PACLITAXEL | | | | |
| (HA) | | | | |
| NAB- | 87 | 9 | 4 | 0 |
| PACLITAXEL- | | | | |
| NFZ | | | | |
| NAB- | 86 | 8 | 2 | 4.27 |
| PACLITAXEL- | | | | |
| NF1 | | | | |

The total impurities and 7-epipaclitaxel generated during storage was analyzed. The results are provided in Table 5.

TABLE 5
Comparison of paclitaxel impurities for NAB-PACLITAXEL
formulations containing recombinant human albumin
or human albumin.

| Storage | | | |
|---|---|---|---|
| Time at | | Paclitaxel Impurities (% of Solution) | |
| 55° C. | Formulations | 7-Epipaclitaxel | Total Impurities |
| 0-time | NAB- | 0.09 | 0.29 |
| | PACLITAXEL | | |
| | (rHA) | | |
| | NAB- | 0.11 | 0.32 |
| | PACLITAXEL | | |
| | (HA) | | |
| | NAB- | 0.08 | 0.28 |
| | PACLITAXEL- | | |
| | NFZ | | |
| | NAB- | 0.19 | 0.35 |
| | PACLITAXEL- | | |
| | NF1 | | |
| 2 Weeks | NAB- | 0.51 | 0.71 |
| | PACLITAXEL | | |
| | (rHA) | | |
| | NAB- | 0.68 | 0.92 |
| | PACLITAXEL | | |
| | (HA) | | |
| | NAB- | 0.36 | 0.56 |
| | PACLITAXEL- | | |
| | NFZ | | |
| | NAB- | 0.53 | 0.68 |
| | PACLITAXEL- | | |
| | NF1 | | |
| 1 Month | NAB- | 0.71 | 0.92 |
| | PACLITAXEL | | |
| | (rHA) | | |
| | NAB- | 0.82 | 1.01 |
| | PACLITAXEL | | |
| | (HA) | | |
| | NAB- | 0.51 | 0.72 |
| | PACLITAXEL- | | |
| | NFZ | | |
| | NAB- | 0.73 | 0.93 |
| | PACLITAXEL- | | |
| | NF1 | | |

### Example 5

In this experiment we evaluated the effect of albumin/paclitaxel ratios (w/w) on the albumin profiles, particle size, and reconstitution time in paclitaxel/albumin nanoparticle compositions prepared using 20% human serum albumin from Grifols (NAB-PACLITAXEL) and 20% recombinant human albumin from Novozyme (NAB-PACLITAXEL-NFZ) as discussed in Example 5. The paclitaxel/albumin nanoparticle formulations were made in accordance with the methods described in Examples 1 and 2. Final albumin/paclitaxel ratios were adjusted by controlling the amount of total albumin added to the formulation.

The albumin profiles of paclitaxel/albumin formulations having albumin/paclitaxel ratios of 4:1, 5:1, 6:1, and 8:1 were analyzed. The results are summarized in Table 6.

TABLE 6
Comparison of albumin isomers for NAB-PACLITAXEL formulations containing different human
albumin to paclitaxel ratios.

| | Albumin Isomers (% of Solution) | | | | | | | |
|---|---|---|---|---|---|---|---|---|
| | | | | | Unnamed | | | |
| | Monomer | | Dimer | | Oligomer | | Polymer | |
| Ratio of | NAB- | NAB- | | NAB- | | NAB- | | NAB- |
| Albumin to | PACLI- | PACLITAXEL- | NAB- | PACLITAXEL- | NAB- | PACLITAXEL- | NAB- | PACLITAXEL- |
| Paclitaxel | TAXEL | NFZ | PACLITAXEL | NFZ | PACLITAXEL | NFZ | PACLITAXEL | NFZ |
| 4:1 | 78 | 74 | 15 | 20 | 3 | 6 | 4.34 | 0 |
| 5:1 | 79 | 79 | 13 | 16 | 3 | 5 | 4.50 | 0 |
| 6:1 | 81 | 82 | 11 | 13 | 3 | 5 | 4.56 | 0 |
| 8:1 | 83 | 86 | 9 | 10 | 2 | 4 | 4.66 | 0 |

The total impurities and 7-epipaclitaxel generated during storage was analyzed. The results are provided in Table 7.

TABLE 7
Comparison of paclitaxel impurities for NAB-PACLITAXEL formulations containing different
human albumin to paclitaxel ratios.

| | | Paclitaxel Impurities (% of Solution) | | | |
|---|---|---|---|---|---|
| | | 7-Epipaclitaxel | | Total Impurities | |
| Storage | Ratio of | | NAB- | | NAB- |
| Time at | Albumin to | NAB- | PACLITAXEL- | NAB- | PACLITAXEL- |
| 55° C. | Paclitaxel | PACLITAXEL | NFZ | PACLITAXEL | NFZ |
| 0 time | 4:1 | 0.09 | 0.06 | 0.32 | 0.23 |
| | 5:1 | 0.10 | 0.06 | 0.27 | 0.19 |
| | 6:1 | 0.09 | 0.06 | 0.26 | 0.19 |
| | 8:1 | 0.11 | 0.06 | 0.33 | 0.24 |
| 2 Weeks | 4:1 | 0.60 | 0.30 | 0.80 | 0.43 |
| | 5:1 | 0.65 | 0.33 | 0.86 | 0.46 |
| | 6:1 | 0.60 | 0.35 | 0.80 | 0.48 |
| | 8:1 | 0.64 | 0.42 | 0.78 | 0.55 |
| 1 Month | 4:1 | 0.84 | n/a | 1.05 | n/a |
| | 5:1 | 0.87 | n/a | 1.13 | n/a |
| | 6:1 | 0.82 | n/a | 1.03 | n/a |
| | 8:1 | 0.83 | n/a | 1.04 | n/a |

Reconstitution time is analyzed for the different formulations, and results are summarized in Table 8.

TABLE 8
Comparison of reconstitution time for NAB-PACLITAXEL
formulations containing different human albumin to paclitaxel ratios.

| | Ratio of | | |
|---|---|---|---|
| Storage | Albumin | | |
| Time at | to | Reconstitution Time (Min:Sec) | |
| 55° C. | Paclitaxel | NAB-PACLITAXEL | NAB-PACLITAXEL-NFZ |
| 0-time | 4:1 | 5:00 | 0:25 |
| | 5:1 | 5:00 | 0:44 |
| | 6:1 | 5:07 | 0:52 |
| | 8:1 | 5:26 | 2:23 |
| 2 | 4:1 | 5:20 | 0:33 |
| Weeks | 5:1 | 5:37 | 0:42 |
| | 6:1 | 5:22 | 0:51 |
| | 8:1 | 7:10 | 3:10 |
| 1 | 4:1 | 5:11 | n/a |
| Month | 5:1 | 5:21 | n/a |
| | 6:1 | 5:30 | n/a |
| | 8:1 | 5:54 | n/a |

Particle sizes were analyzed for the different formulations, and results are summarized in Table 9.

TABLE 9
Comparison of particle size for NAB-PACLITAXEL formulations containing different human
albumin to paclitaxel ratios.

| | | Particle Size (nm) | | | | | |
|---|---|---|---|---|---|---|---|
| | | Mean Size | | <5% | | <95% | |
| Storage | Ratio of | | NAB- | | NAB- | | NAB- |
| Time at | Albumin to | NAB- | PACLITAXEL- | NAB- | PACLITAXEL- | NAB- | PACLITAXEL- |
| 55° C. | Paclitaxel | PACLITAXEL | NFZ | PACLITAXEL | NFZ | PACLITAXEL | NFZ |
| 0-time | 4:1 | 119 | 121 | 76 | 78 | 173 | 172 |
| | 5:1 | 117 | 119 | 75 | 77 | 170 | 169 |
| | 6:1 | 120 | 120 | 78 | 78 | 175 | 171 |
| | 8:1 | 115 | 120 | 75 | 78 | 165 | 170 |
| 2 Weeks | 4:1 | 125 | 118 | 80 | 77 | 179 | 169 |
| | 5:1 | 123 | 116 | 80 | 74 | 176 | 167 |
| | 6:1 | 126 | 118 | 82 | 77 | 178 | 170 |
| | 8:1 | 121 | 116 | 79 | 72 | 171 | 168 |
| 1 Month | 4:1 | 125 | n/a | 79 | n/a | 183 | n/a |
| | 5:1 | 124 | n/a | 79 | n/a | 177 | n/a |
| | 6:1 | 122 | n/a | 79 | n/a | 175 | n/a |
| | 8:1 | 120 | n/a | 78 | n/a | 171 | n/a |

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope described herein.

What is claimed is:

1. A pharmaceutical composition comprising nanoparticles comprising albumin and paclitaxel, wherein no greater than about 2.4% of the total albumin in the pharmaceutical composition is in the form of polymers.

2. The pharmaceutical composition of claim 1, wherein at least about 80% of the total albumin in the pharmaceutical composition is in the form of monomers.

3. The pharmaceutical composition of claim 2, wherein at least about 92% of the total albumin in the pharmaceutical composition is in the form of monomers.

4. The pharmaceutical composition of claim 1, wherein at least about 60% of the monomeric albumins in the pharmaceutical composition have a free thiol group.

5. The pharmaceutical composition of claim 1, wherein at least about 60% of the monomeric albumin in the pharmaceutical composition have a blocked thiol group.

6. The pharmaceutical composition of claim 1, wherein no greater than about 10% of total albumin in the pharmaceutical composition is in the form of dimers.

7. The pharmaceutical composition of claim 1, wherein no greater than about 3% of total albumin in the pharmaceutical composition is in the form of oligomers.

8. The pharmaceutical composition of claim 1, wherein the pharmaceutical composition is substantially free of albumin lacking C-terminal Leu and albumin lacking N-terminal Asp-Ala.

9. The pharmaceutical composition of claim 1, wherein the albumin in the pharmaceutical composition has a glycosylation profile that is different from that of native albumin obtained from a human.

10. The pharmaceutical composition of claim 1, wherein the albumin in the pharmaceutical composition has no glycosylation.

11. The pharmaceutical composition of claim 1, wherein the c pharmaceutical composition is substantially free of fatty acids.

12. The pharmaceutical composition of claim 1, wherein the pharmaceutical composition is substantially free of caprylate.

13. The pharmaceutical composition of claim 1, wherein the pharmaceutical composition is substantially free of tryptophan.

14. The pharmaceutical composition of claim 1, wherein the pharmaceutical composition is substantially free of a blood component.

15. The pharmaceutical composition of claim 1, wherein at least about 80% of the total albumin in the pharmaceutical composition is not associated with the nanoparticles.

16. The pharmaceutical composition of claim 1, wherein the nanoparticles comprise paclitaxel coated with albumin.

17. The pharmaceutical composition of claim 1, wherein the nanoparticles in the pharmaceutical composition have an average diameter of no greater than about 200 nm.

18. The pharmaceutical composition of claim 1, wherein the weight ratio of the albumin and the paclitaxel in the composition is about 9:1 to about 1:1.

19. The pharmaceutical composition of claim 1, wherein the albumin is human albumin.

20. A commercial batch of the pharmaceutical composition of claim 1.

21. A method of treating a cancer in an individual, comprising administering to the individual an effective amount of the pharmaceutical composition of claim 1.

22. The method of claim 21, wherein the individual is human.
